SEQUENCE LISTING (4 sequences)

SEQ ID NO: 1 - protein, 33 aa, artificial
  amino acid sequence of the anti-angiogenic peptide 33mer Anginex
ANIKLSVQMKLFKRHLKWKIIVKLNDGRELSLD

SEQ ID NO: 2 - protein, 6 aa, artificial
  amino acid sequence fragment of dibenzofuran (DBF)-based mimetic 6DBF7
SVQMKL

SEQ ID NO: 3 - protein, 7 aa, artificial
  amino acid sequence fragment of dibenzofuran (DBF)-based mimetic 6DBF7
IIVKLND

SEQ ID NO: 4 - protein, 135 aa, Homo sapiens
MACGLVASNLNLKPGECLRVRGEVAPDAKSFVLNLGKDSNNLCLHFNPRFNAHGDANTIVCNSKDGGAWGTEQREAVFPFQPGSVAEVCITFDQANLTVKLPDGYEFKFPNRLNLEAINYMAADGDFKIKCVAFD

(19) United States
(12) Patent Application Publication
(10) Pub. No.: US 2014/0121278 A1
(43) Pub. Date: May 1, 2014

(54) ANTI-TUMOR AGENT OTX-008 TARGETS HUMAN GALECTIN-1

(71) Applicant: REGENTS OF THE UNIVERSITY OF MINNESOTA, St. Paul, MN (US)

(72) Inventor: KEVIN H. MAYO, Minneapolis, MN (US)

(73) Assignee: Regents of the University of Minnesota, St. Paul, MN (US)

(21) Appl. No.: 13/666,085

(22) Filed: Nov. 1, 2012

(51) Int. Cl. A61K 31/16; A61P 35/00; C12N 5/09

(52) U.S. Cl. 514/616; 435/375

(57) ABSTRACT

Disclosed herein are compositions comprising a galectin-1-targeting compound in a therapeutically effective composition for treating cancer. In an aspect, a galectin-1-targeting compound is OTX-008. Also disclosed herein are methods of making and using such compositions.

### GOVERNMENT FUNDING

This invention was made with government support under Grant No. CA096090, awarded by the National Institutes of Health. The Government has certain rights in the invention.

### BACKGROUND

Galectins are a family of beta-galactoside binding lectins containing homologous carbohydrate recognition domains (CRDs). To date, fifteen mammalian galectin family members have been identified. These lectins are known to play a key role in many pathological states, including, for example, autoimmune diseases, allergic reactions, inflammation, tumor cell metastasis, atherosclerosis, and diabetic complications. Galectin-1 contributes to many events associated with cancer biology, including, for example, tumor transformation, cell cycle regulation, apoptosis, cell adhesion, migration and inflammation. Galectin-1 is abundantly secreted by many types of malignant tumor cells (Ito et al., 2012, Cancer Metastasis Rev; 31(3-4):763-778) and evidence indicates that galectin-1 plays a role in tumor evasion of immune responses (Rabinovich, 2005, Br J Cancer; 92(7):1188-1192).

Galectin-1 was first described as a determinant factor in cancer cell adhesion but was ultimately recognized as a multifunctional protein involved in various aspects of tumorigenesis, cell-extracellular matrix and cell-cell interactions, cell migration, angiogenesis, and immune surveillance escape (Danguy et al., 2002, Biochim Biophys Acta; 1572:285-293). The up-regulation of galectin-1 is associated with poor prognosis and acquisition of a metastatic phenotype in several tumor models. Consistently, exposure to exogenous galectin-1 has been shown to promote metastasis by positively regulating cell migration and invasiveness. Galectin-1 expression has been examined in several malignant tumors. Correlations between galectin-1 expression, tumor invasiveness and lymph node metastasis have been demonstrated in hepatocarcinoma, breast cancer, neuroblastoma, lung adenocarcinoma, and head and neck squamous cell carcinomas. Cancer cells that secrete galectin-1 may form extracellular lattices, which facilitate growth factor/receptor and cell/matrix interactions. Galectin-1 has been was shown to enhance VEGFR-dependent angiogenesis through interaction with neuropilin-1. The binding of galectin-1 to neuropilin-1 enhances VEGFR2 phosphorylation and stimulates the activation of the MAPK pathways. Moreover, galectin-1 is highly up-regulated in tumor-activated endothelial cells and is crucial for activated endothelial cells to adhere to, and to migrate on the extracellular matrix. More recently, reports support an interaction between intracytoplasmic galectin-1 and Ras-dependent signaling cascades. Galectin-1 has been shown to bind oncogenic H-Ras, increase membrane associated Ras-GTP facilitated Raf activation, inhibit caveolin formation, and promote dissociation of Ras anchorage from the plasma membrane toward the endoplasmic Golgi and reticulums. These processes were shown to be critical in the oncogenic role of Ras as well as in downstream activation of Raf-1 and ERK1/2.

Galectin-1 is involved in pathological disorders like tumor endothelial cell adhesion and migration and therefore presents a relevant target for therapeutic intervention against cancer. Thus, there is a need for therapeutic agents that target and modulate galectin-1 mediated pathways.

### SUMMARY OF THE INVENTION

The present invention includes methods of inhibiting a pathway associated with galectin-1 in a mammalian cell, wherein galectin-1 has a front face β-sheet and a back face β-sheet, the method including contacting a mammalian cell with an effective amount of a galectin-1-targeting compound, wherein the galectin-1-targeting compound has an affinity for the back face β-sheet of galectin-1.

The present invention includes methods of treating cancer in a subject in need thereof, the method including administering a therapeutically effective amount of a compound having an affinity for a back face β-sheet of galectin-1 to the subject in need thereof.

The present invention includes methods of inhibiting the galectin-1/semaphorin-3A system in a cancer cell, the method including contacting the cell with an effective amount of a galectin-1-targeting compound, wherein the compound binds to galectin-1 on at least one site other than the carbohydrate binding site.

The present invention includes methods of allosterically inhibiting the binding of a glycan carbohydrate to galectin-1 at a site other than the canonical β-galactoside carbohydrate binding site, the method including contacting the galectin-1 with OTX-008 or a derivative thereof, OTX-008 having the formula

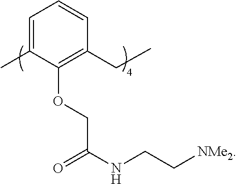

The present invention includes methods of inhibiting proliferation in galectin-1 positive cells, the method including contacting the galectin-1 positive cells with OTX-008 or a derivative thereof, OTX-008 having the formula

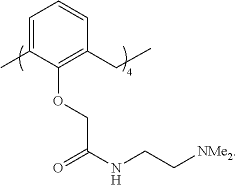

The present invention includes methods of inhibiting the binding of galectin-1 to cell surface glycans and/or inhibiting galectin-1 mediated cell agluttination, the method including contacting the cells with OTX-008 or a derivative thereof, OTX-008 having the formula

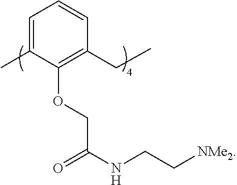

In some aspects of the methods of the present invention, a galectin-1-targeting compound additionally binds to the front face β-sheet.

In some aspects of the methods of the present invention, a galectin-1-targeting compound does not bind to the front face β-sheet.

In some aspects of the methods of the present invention, a galectin-1-targeting compound binds to galectin-1 on at least one site other than the carbohydrate binding site.

In some aspects of the methods of the present invention, a galectin-1-targeting compound attenuates the binding of lactose to the front face β-sheet.

In some aspects of the methods of the present invention, a galectin-1-targeting compound attenuates the binding of the galectin-1 to one or more cell surface glycans.

In some aspects of the methods of the present invention, a galectin-1-targeting compound binds to galectin-1 with a K_{d} in the range of about 1 μM to about 100 μM.

In some aspects of the methods of the present invention, inhibiting galectin-1 activity includes contacting a cell with a therapeutically effective amount of a galectin-1 targeting compound.

In some aspects of the methods of the present invention, a galectin-1-targeting compound contacts one or more residues of a galectin-1 selected from residues corresponding to residues 6, 7, 8, 9, 10, 14, 15, 16, 17, 89, 90, 91, and/or 92 of the human galectin-1 sequence set forth in SEQ ID NO:4.

In some aspects of the methods of the present invention, a galectin-1-targeting compound contacts one or more residues of human galectin-1 selected from residues 6, 7, 8, 9, 10, 14, 15, 16, 17, 89, 90, 91, and/or 92 of the human galectin-1 sequence set forth in SEQ ID NO:4.

In some aspects of the methods of the present invention, a galectin-1 targeting compound is OTX-008 or a derivative thereof, OTX-008 having the formula

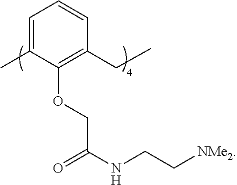

In some aspects of the methods of the present invention, the method includes contacting one or more cancerous cells in a subject with cancer with a therapeutically effective amount of the galectin-1 targeting compound.

In some aspects of the methods of the present invention, inhibiting a pathway associated with galectin-1 includes inhibiting growth of the cancer cell, proliferation of the cancer cell, and/or tumor metastasis.

In some aspects of the methods of the present invention, the treatment of the cancer includes at least one of slowing the growth of the cancer, inhibiting the spread of a tumor associated with the cancer, inhibiting the spread of one or more metastases associated with the cancer, reducing the size of a tumor associated with the cancer, and/or inhibiting the recurrence of cancer treated previously.

In some aspects of the methods of the present invention, the galectin-1 targeting compound is administered by at least one route selected from the group consisting of intravenously, subcutaneously, intradermally, parenterally, and intramuscularly.

In some aspects of the methods of the present invention, a galectin-1 targeting compound is administered in a single dose.

In some aspects of the methods of the present invention, a galectin-1 targeting compound is administered in two or more doses.

In some aspects of the methods of the present invention, a galectin-1 targeting compound is administered in combination with at least one other mode of therapy. In some aspects, other modes of therapy compound include is radiotherapy, chemotherapy, and surgery, or combinations thereof.

In some aspects of the methods of the present invention, a therapeutically effective amount of the galectin-1 targeting compound includes a pharmaceutical composition.

In some aspects of the methods of the present invention, a galectin-1 targeting compound includes OTX-008 or a derivative thereof, OTX-008 having the formula

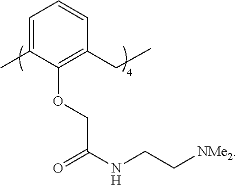

In some aspects of the methods of the present invention, a galectin-1 targeting compound is administered to a subject by at least one route of intravenously, subcutaneously, intradermally, parenterally, and/or intramuscularly.

In some aspects of the methods of the present invention, a galectin-1 targeting compound is administered as a pharmaceutical composition to a subject by at least one route of intravenously, subcutaneously, intradermally, parenterally, and/or intramuscularly.

In some aspects of the methods of the present invention, a cancer includes ovarian cancer, squamous cell carcinoma, a cancer of the digestive system, stomach cancer, liver cancer, colon cancer, a cancer of the thyroid, a cancer of the endometrium, adenocarcinoma of the endometrium, uterine cancer, uterine adenocarcinoma, a uterine smooth muscle tumor, breast cancer, prostate cancer, bladder cancer, a head cancer, a neck cancer, a glioma, a kidney cancer, pancreatic cancer, pancreatic ductal adenocarcinoma, nonsmall-cell lung cancer, or melanoma.

In some aspects of the methods of the present invention, a galectin-1 targeting compound may be administered to a subject in a dose selected from about 1 microgram to about 1,000 micrograms, about 25 micrograms to about 500 micrograms, about 50 to about 250 micrograms, about 2 micrograms to about 100 micrograms, about 5 micrograms to about 75 micrograms, about 10 micrograms to about 50 micrograms, or about 20 micrograms to about 40 micrograms.

In some aspects of the methods of the present invention, OTX-008 may be administered to a subject in a dose selected from about 1 microgram to about 1,000 micrograms, about 25 micrograms to about 500 micrograms, about 50 to about 250 micrograms, about 2 micrograms to about 100 micrograms, about 5 micrograms to about 75 micrograms, about 10 micrograms to about 50 micrograms, or about 20 micrograms to about 40 micrograms.

### BRIEF DESCRIPTION OF THE DRAWINGS

FIG. 1 depicts the amino acid sequence of the anti-angiogenic peptide 33mer Anginex (SEQ ID NO:1), the dibenzofuran (DBF)-based mimetic 6 DBF7 (the amino acid sequences SVQMKL (SEQ ID NO:2) and IIVKLND (SEQ ID NO:3) covalently bonded with dibenzofuran (DBF)), and two separate representations of the non-peptidic calix[4]arene-based compound OTX-008 (“0018”).

FIG. 2 depicts an overlay of two ¹H-¹⁵N HSQC spectral expansions for ¹⁵N-enriched gal-1 (1 mg/ml), alone and in the presence of 400 μM OTX-008 (0118). Resonances are labeled with assignments reported previously (Nesmelova et al, 2008, Biomol NMR Assign; 2(2):203-205). The insert shows the concentration of unbound or free OTX-008 ligand (“0018”) calculated by subtracting the estimated fraction bound of OTX-008 from the total concentration of OTX-008 in solution.

FIGS. 3A and 3B depict HSQC maps for resonance broadening (FIG. 3A) and chemical shift (FIG. 3B) for OTX-008 binding to gal-1 (1 mg/ml). Fractional changes in gal-1 resonance intensities observed for gal-1 in the presence of OTX-008 (170 μM) are shown versus the amino acid sequence of gal-1 in FIG. 3A. A value of 1 indicates that the resonance associated with that particular residue is no longer apparent, and a value of zero indicates no change in resonance intensity. Chemical shift changes to ¹⁵N-gal-1 resonances in the presence of OTX-008 are shown versus the amino acid sequence of gal-1 (FIG. 3B).

FIG. 4 depicts the dimer structure of gal-1 (PDB access code 1gzw). Residues that are most broadened and chemically shifted as indicated in FIG. 2 are highlighted. The proposed OTX-008 (“0118”) binding region on gal-1 is indicated by a circle. Three faces are depicted: the lactose binding front face, the back side opposite the lactose binding face, and a side view between the two faces looking on edge through the β-sandwich. A stick structure of OTX-008 is shown to scale in the insert at the bottom of the figure.

FIGS. 5A-5D depict lactose titration curves for four gal-1 residues (N39 (FIG. 5A), F79 (FIG. 5B), R73 (FIG. 5C), and S29 (FIG. 5D)). ¹H, ¹⁵N-weighted chemical shift differences (Δδ=[(δ¹H)²+0.15(δ¹⁵N)²]^{1/2}) were determined from HSQC spectra acquired with ¹⁵N-gal-1 (100 μM) in the absence () and presence (▪) of OTX-008 (“0118”) in a molar ratio of approximately 1:6 (gal-1:OTX-008). The total lactose concentration is shown in these plots. Titration curves were fit with Gaussian/sigmoidal functions, and Kd values were determined by taking the total lactose concentration at 50% Δδ minus the concentration of bound lactose (i.e. half of the gal-1 concentration=50 μM).

FIGS. 6A and 6B depict the results of flow cytometry used to assess the effects of OTX-008 (“0118”) on FITC-gal-1 binding to splenocytes (CD4+ and CD8+ leukocytes and CD31+ endothelial cells). FIG. 6A illustrates the binding of FITC-gal-1 (0.1 μM or 0.2 μM) to these cells as a function of OTX-008 concentration. FIG. 6B illustrates the binding to CD4+ and CD8+ leukocytes and CD31+ endothelial cells derived from male or female gal-1−/− null mice.

FIG. 7 depicts Jurkat E6.1 cells agglutination as induced by addition of 1 μM gal-1 and incubated at 22° C. on plastic, round bottom chamber slides (Nunc, Naperville, Ill., USA), in medium alone or in the presence of 20 μM OTX-008 (“0118”).

FIGS. 8A-8C illustrate the concentration of OTX-008 (“0118”) required for inhibiting cell viability is proportional to Gal-1 expression. FIG. 8A illustrates the anti-viability effects (expressed as % change in cell numbers over time) of OTX-008 using the MTT assay in human cancer cell lines. FIG. 8B illustrates cancer cells that are sensitive to OTX-008 expressed lower galectin-1 protein expression as determined using Western Blot analysis. FIG. 8C illustrates that concentration of OTX-008, as established by plotting the IC50 in cancer cells, is proportional to their relative LGALS1 mRNA expression as assessed by qRT-PCR.

### DETAILED DESCRIPTION

With the present invention, NMR spectroscopy was used to establish that galectin-1 is the molecular target of the calixarene OTX-0008, demonstrating that OTX-0008 interacts with this lectin at a site other than its carbohydrate binding site. Further, the present invention demonstrates that the calixarene OTX-0008 attenuates galectin-1 binding to cell surface glycans, attenuates galectin1-mediated cell agglutination, and inhibits cell proliferation in a dose-dependent manner that is correlated with the expression of galectin-1 on the cell surface.

Disclosed herein are methods of targeting and inhibiting galectin-1, including galectin-1 activity and/or expression, including expression of galectin-1 mRNA and/or galectin-1 protein. In part, disclosed herein are methods of inhibiting galectin-1 by administering a galectin-1-targeting compound, including, but not limited to the calixarene OTX-008.

In an embodiment, binding of such a galectin-1-targeting compound to galectin-1 attenuates the binding of galectin-1 to one or more cell surface glycans.

Galectin-1 (gal-1) is a rather ubiquitous carbohydrate-binding protein (binding to β-galactoside groups on various cell surface receptors) that is over-expressed in many different types of cancers. Increased galectin-1 expression by tumor and connective tissue supporting the tumor often correlates with the aggressiveness of the tumor and the acquisition of a metastatic phenotype. In preclinical studies, galectin-1 has been shown to play a role in tumor transformation, tumor cell proliferation, cell aggregate, adhesion, migration, apoptosis, and immunoregulation. Galectin-1 is often found to be highly elevated in tumor stroma in several cancers including breast, colon, prostate and ovarian. The galectin-1 targeting compounds and compositions thereof of the present disclosure may be administered to a patient for the treatment of a cancer in which the cancerous cells express a high level of galectin-1. A compound, conjugate, or composition of the present disclosure may be administered to a patient for the treatment of a cancer in which the cancerous cells express low levels of galectin-1. A compound, conjugate, or composition of the present disclosure may be administered to a patient for the treatment of a cancer in which cancerous cells do not express galectin.

In an embodiment, the galectin-1-targeting compound binds to the back face β-sheet of galectin-1. In an embodiment, binding of a galectin-1-targeting compound to galectin-1 attenuates the binding of a carbohydrate, such as, for example, lactose or a cell surface glycan, to the front face β-sheet of galectin-1. Galectins are a structurally related family of lectins defined by their affinity for β-galactoside glycans. Galectin-1 has a β-sandwich structure that is comprised of 11 β-strands, with the front face β-sheet (anti-parallel running β-strands 1, 3, 10, 4, 5, and 6) containing the lactose binding site, and the back face β-sheet comprising β-strands 7, 8, 9, 2, and 11. β-strands 1 and 11 interact to form the galectin-1 dimer interface.

Galectins bind to β-galactosides at a canonical site in the carbohydrate recognition domain (CRD) (Barondes et al., 1994, Cell; 76(4):597-598). Because antagonists of galectins have therapeutic potential as anti-inflammatory agents and anticancer agents, efforts have been made to design galectin antagonists. However, to date, reported galectin antagonists are mostly β-galactoside analogues and glycomimetics that target the canonical β-galactoside carbohydrate binding site. A galectin-1-targeting compound of the present invention may interact with galaectin-1 by binding to the lectin at a site other than the canonical carbohydrate binding site. For example, it may bind to one of more of the β-strands 7, 8, 9, 2, and/or 11 that form the back face β-sheet of galectin-1.

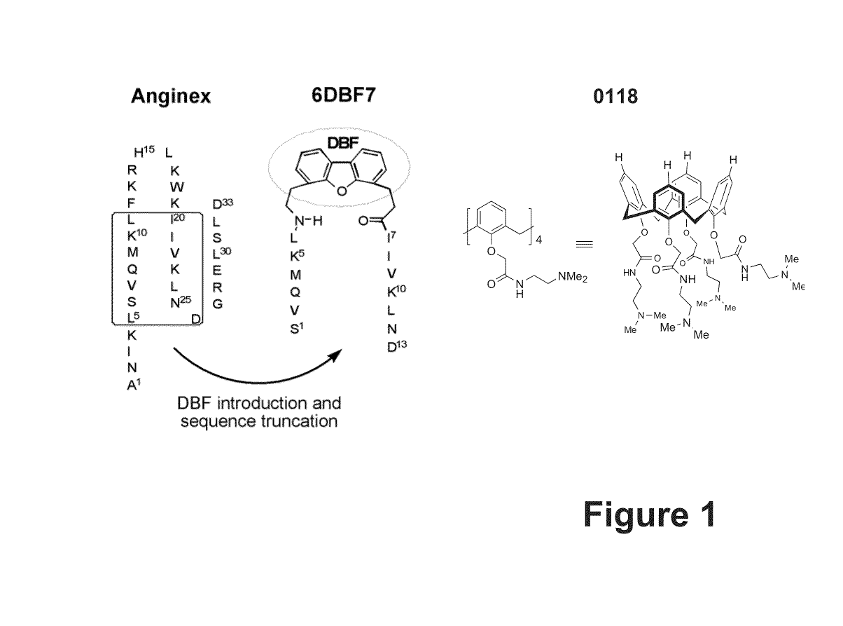

In some embodiments of the methods described herein, a galectin-1-targeting compound is OTX-008 (also referred to as compound 40, 0118, KM0118, or PTX-008), a derivative or intermediate thereof. The synthesis, structure, properties, and activities of OTX-008 are described in more detail in WO 2006/042104; US 2008/0300164; Dings et al., 2006, J Natl Cancer Inst; 98(13):932-936; Chen et al., 2006, J Med Chem; 49(26):7754-7765; and Dings et al., 2008, Cancer Lett; 265(2):270-280, each of which is incorporated by reference herein in its entirety. As shown in FIG. 1, OTX-008 has the formula

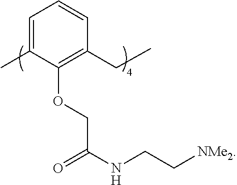

A galectin-1-targeting compound may exist in unsolvated forms as well as solvated forms, including hydrated forms. In general, the solvated forms are equivalent to unsolvated forms and are encompassed within the scope of the present invention. Certain compounds of the present invention may exist in multiple crystalline or amorphous forms. In general, all physical forms are equivalent for the uses contemplated by the present invention and are intended to be within the scope of the present invention.

A galectin-1-targeting compound may possess asymmetric carbon atoms (optical centers) or double bonds; the racemates, diastereomers, geometric isomers and individual isomers are encompassed within the scope of the present invention. The graphic representations of racemic, ambiscalemic and scalemic or enantiomerically pure compounds used herein follow those described by Maehr, 1985, J Chem Ed; 62:114-120.

A galectin-1-targeting compound can exist in particular geometric or stereoisomeric forms. The invention contemplates all such compounds, including cis- and trans-isomers, (−)- and (+)-enantiomers, (R)- and (S)-enantiomers, diastereomers, (D)-isomers, (L)-isomers, the racemic mixtures thereof, and other mixtures thereof, such as enantiomerically or diastereomerically enriched mixtures, as falling within the scope of the invention. Additional asymmetric carbon atoms can be present in a substituent such as an alkyl group. Any such isomers, as well as mixtures thereof, may be used in the methods described herein.

Also disclosed herein are methods of inhibiting a galectin-1-associated pathway in a mammalian cell by contacting the cell with one or more galectin-1 targeting compounds.

Also disclosed herein are compositions and methods for treating cancer in mammals by the administration of one or more galectin-1 targeting compounds. In an aspect, compositions and methods are provided for treatment of cancer in a mammal, including treatment of tumors, via inhibition of a galectin-1 associated pathway. In an aspect, compositions and methods are provided for treatment of cancer in a mammal, including treatment of tumors, via modulation of galectin-1. In some aspects, the administration of a galectin-1 targeting compound may demonstrate antiproliferative and/or anti-invasive effects on cancer cells and human tumors.

As demonstrated herein, a galectin-1 targeting compound, such as, for example, OTX-008, may demonstrate an effect in cancer cells expressing low levels of galectin-1, high levels of epithelial markers such as E-cadherin, and low levels of mesenchymal markers such as vimentin.

In an aspect, a galectin-1 targeting compound, such as, for example, OTX-008, may down-regulate MAPK and AKT/mTOR survival pathways and galectin-1 protein expression. In another aspect, induces MAPK-dependent G2/M-transition inhibition in vitro and inhibits tumor growth, angiogenesis, and metastasis in vivo. In an embodiment, encompassed herein are methods of targeting galectin-1 as a target for anticancer therapy. In an embodiment, encompassed herein are methods of using a galectin-1 targeting compound, such as, for example, OTX-008, to target galectin-1 as a target for anticancer therapy. In an embodiment, encompassed herein are methods of treating cancers involving galectin-1-overexpressing cells. In an embodiment, encompassed herein are methods of treating cancers involving galectin-1-overexpressing cells by contacting galectin-1 overexpressing cells with a galectin-1 targeting compound, such as, for example, OTX-008, in order to decrease or diminish the levels of galectin-1 mRNA and/or protein in such cells.

In an embodiment, a galectin-1-targeting compound demonstrates at least one of the biological activities described herein. One example of a galectin-1 targeting compound is OTX-008. In an embodiment, OTX-008 has at least one of the biological activities described herein. The biological activity of a compound can be determined, for example, as described herein or by methods well known to one of skill in the art. As will be understood by the skilled artisan viewing the disclosure encompassed herein, an activity of a galectin-1 targeting compound such as, for example, OTX-008, is one which arises through modulation of galectin-1, as described herein. Such a biological activity may include, but is not limited to, binding to galectin-1. Binding to galectin-1 includes binding to at least one amino acid residue of galectin-1. In some embodiments, binding to galectin-1 may include binding to the back face beta sheet of galectin-1. In some embodiments, binding to galectin-1 may include binding to both the back face beta sheet of galectin-1 and to the front face beta sheet of galectin-1. In some embodiments, binding to galectin-1 may include binding to the back face beta sheet of galectin-1 but not binding to the front face beta sheet of galectin-1.

Galectin-1 has a β-sandwich structure that is comprised of 11 β-strands, with the front face β-sheet (anti-parallel running β-strands 1, 3, 10, 4, 5, and 6) containing the lactose binding site (also referred to herein as the canonical β-galactoside carbohydrate binding site, the canonical site of the carbohydrate recognition domain (CRD)), and the back face β-sheet comprising β-strands 7, 8, 9, 2, and 11. β-strands 1 and 11 interact to form the galectin-1 dimer interface.

The back face of galectin-1 may include amino acids residues corresponding to residues 6, 7, 8, 9, 10, 14, 15, 16, 17, 89, 90, 91, and/or 92 of a galectin-1. The back face of galectin-1 may include a region of galectin-1 formed by amino acid residues 6-10, amino acid residues 14-17, and/or amino acid residues 89-92 of galectin-1.

In some aspects, galectin-1 is human galectin-1 having the amino acid sequence MACGLVASNL NLKPGECLRV RGEVAPDAKS FVLNLGKDSN NLCLHFNPRF NAHGDANTIV CNSKDGGAWG TEQREAVFPF QPGSVAEVCI TFDQANLTVK LPDGYEFKFP NRLNLEAINY MAADGDFKIK CVAFD (SEQ ID NO:4). See also, National Center for Biotechnology Information (NCBI) Accession NP_{—}002296, which is hereby incorporated by reference in its entirety.

In some embodiments, a galectin-1-targeting compound is not OTX-008. In an embodiment, a composition is provided, the composition comprising at least two non-OTX-008 galectin-1-targeting compounds. In an embodiment, a composition is provided, the composition comprising OTX-008 and at least one non-OTX-008 galectin-1-targeting compound. In an aspect of an embodiment, the non-OTX-008 compound is a calixarene. In an embodiment, a non-OTX-008 compound binds to the back face beta sheet of galectin-1. In an embodiment, a non-OTX-008 compound binds to the front face beta sheet of galectin-1. In an embodiment, a non-OTX-008 compound binds to the back face beta sheet and the to the front face beta sheet of galectin-1.

With the methods described herein, a galectin-1-targeting compound may target galectin-1. “Targeting” of galectin-1, as the term is used herein, may include the direct physical contact of a compound with—or affinity for—galectin-1, and/or any indirect effect of the compound on galectin-1, including down-regulation of galectin-1 expression via the compound's interaction with a non-galectin-1 moiety. OTX-008 “targeting” of galectin-1, as the term is used herein, may include the direct physical contact of OTX-008 with—or affinity for—galectin-1, as well as any indirect effect of OTX-008 on galectin-1, including down-regulation of galectin-1 expression via OTX-008 interaction with a non-galectin-1 moiety.

With the methods described herein, a galectin-1-targeting compound may modulate galectin-1 or galectin-1 activity. “Modulation” of galectin-1 or galectin-1 activity, as the term is used herein, may include, but is no limited to, the up- or down-regulation of galectin-1 expression, and the up- or down-regulation of galectin-1 activity, and/or causing galectin-1 to demonstrate a different, non-typical, or new activity or cellular role, whether or not the different, non-typical, or new activity or role also results in the up- or down-regulation of galectin-1 expression, or the up- or down-regulation of galectin-1 activity.

As the term is used herein, “galectin-1 activity” refers to an end result or process resulting either directly or indirectly from the structure, location, catalysis (whether non-enzymatic or enzymatic), or intermolecular interactions of galectin-1 protein or nucleic acid. As disclosed in detail elsewhere herein, galectin-1 is involved in numerous inter- and intra-cellular metabolic pathways in various mammalian cells, and the role and function of galectin-1 in these metabolic pathways are encompassed by the term “galectin-1 activity.”

With the methods described herein, a galectin-1-targeting compound may alter a galectin-1 pathway. A “galectin-1-associated pathway” is any cellular or metabolic pathway involving galectin-1 protein or nucleic acid. For example, galectin-1-associated pathways include, but are not limited to, the galectin-1/semaphorin-3A system in cancer cells, pathways involving ERK1/2-, AKT-, and S6-phosphorylation, the MAPK pathway, and pathways involving p-weel, among others. A galectin-1-associated pathway may be demonstrated, as disclosed in detail herein in the experimental examples, by in vivo or in vitro assays employing a known galectin-1-targeting compound.

Assays which can be used to demonstrate a galectin-1-associated pathway include, but are not limited to, delay of tumor growth in A2780-1A9 human ovarian cancer and SQ20B human squamous cell head-and-neck cancer xenograft models, decreased galectin-1 protein expression and translocation of galectin-1 from the cytoplasm to the nucleus in vitro, and decreasing galectin-1 expression and decreasing Ki67 expression in vivo, in association with a demonstration of antiproliferative effects in cultured cells. The metabolic and signaling pathways involved in such assays are well-known. One or more such galectin-1-associated pathway effects may be assayed in one or more cancer cells lines, including, but not limited to HT29, HCT116, COL0205, or HCC2998 (human colon cancer cell lines), MCF7, MDA-MB-435, SKBR3, or ZR-75-1 (human breast cancer cell lines), OVCAR3, IGROV1, or SKOV3 (human ovarian cancer cell lines), HOP62 or HOP92 (human lung cancer cell lines), PC3 or DU145 (human prostate cancer cell lines), SCC61, SQ20B, or HEP2 (human head and neck cancer cell lines), SK-HEP1 (a human hepatocellularcarcinoma cancer cell line), or CAKI1 (a human renal cancer cell line). See, for example, Astorgues-Xerri et al., 2009, Molecular Cancer Therapies; Volume 8, Issue 12, Supplement 1, Abstract B166.

Assays which can be used to demonstrate a galectin-1-associated pathway include, but are not limited to, any of the assays or cell lines described in PCT/EP2012/055902, which is herein incorporated by reference in its entirety.

“Treat”, “treating”, and “treatment”, etc., as used herein, refer to any action providing a benefit to a patient afflicted with a disease, including improvement in the condition through lessening or suppression of at least one symptom, delay in progression of the disease, prevention or delay in the onset of the disease, etc. Treating can include therapeutic and/or prophylactic treatments. “Treating a disorder,” as used herein, is not intended to be an absolute term. Treatment may lead to an improved prognosis or a reduction in the frequency or severity of symptoms. Desirable effects of treatment include preventing occurrence or recurrence of disease, alleviation of symptoms, diminishment of any direct or indirect pathological consequences of the disease, decreasing the rate of disease progression, amelioration or palliation of the disease state, and remission or improved prognosis. Likewise, the term “preventing,” as used herein, is not intended as an absolute term. Instead, prevention refers to delay of onset, reduced frequency of symptoms, or reduced severity of symptoms associated with a disorder. Prevention therefore refers to a broad range of prophylactic measures that will be understood by those in the art. In some circumstances, the frequency and severity of symptoms is reduced to non-pathological levels. In some circumstances, the symptoms of an individual receiving the compositions of the invention are only 90, 80, 70, 60, 50, 40, 30, 20, 10, 5 or 1% as frequent or severe as symptoms experienced by an untreated individual with the disorder.

The efficacy of treatment of a cancer may be assessed by any of various parameters well known in the art. This includes, but is not limited to, determinations of a reduction in tumor size, determinations of the inhibition of the growth, spread, invasiveness, vascularization, angiogenesis, and/or metastasis of a tumor, determinations of the inhibition of the growth, spread, invasiveness and/or vascularization of any metastatic lesions, determinations of tumor infiltrations by immune system cells, and/or determinations of an increased delayed type hypersensitivity reaction to tumor antigen. The efficacy of treatment may also be assessed by the determination of a delay in relapse or a delay in tumor progression in the subject or by a determination of survival rate of the subject, for example, an increased survival rate at one or five years post treatment. As used herein, a relapse is the return of a tumor or neoplasm after its apparent cessation, for example, such as the return of leukemia.

Toxicity and therapeutic efficacy of the compounds, conjugates, and compositions of the present invention can be determined by standard pharmaceutical procedures in cell cultures or experimental animals, for example, for determining the LD50 (the dose lethal to 50% of the population) and the ED50 (the dose therapeutically effective in 50% of the population). The dose ratio between toxic and therapeutic effects is the therapeutic index and it can be expressed as the ratio between LD50 and ED50. Compounds that exhibit high therapeutic indices are preferred. The data obtained from cell culture assays and animal studies can be used in formulating a range of dosage for use in humans. The dosage of such compounds lies preferably within a range of circulating concentrations that include the ED50 with little or no toxicity. The dosage can vary within this range depending upon the dosage form employed and the route of administration utilized. The exact formulation, route of administration and dosage can be chosen by the individual physician in view of the patient's condition.

Therapeutically effective concentrations and amounts may be determined for each application herein empirically by testing the compounds in known in vitro and in vivo systems, such as those described herein, dosages for humans or other animals may then be extrapolated therefrom.

It is understood that the precise dosage and duration of treatment is a function of the disease being treated and may be determined empirically using known testing protocols or by extrapolation from in vivo or in vitro test data. It is to be noted that concentrations and dosage values may also vary with the severity of the condition to be alleviated. It is to be further understood that for any particular subject, specific dosage regimens should be adjusted over time according to the individual need and the professional judgment of the person administering or supervising the administration of the compositions, and that the concentration ranges set forth herein are exemplary only and are not intended to limit the scope or practice of the claimed compositions and methods.

The term “pharmaceutically acceptable,” as used herein with respect to a compound or composition, refers to a form of the compound or composition that can increase or enhance the solubility or availability of the compound in a subject, in order to promote or enhance the bioavailability of the compound or composition. In an aspect, the disclosure herein also encompasses pharmaceutically acceptable, hydrates, solvates, stereoisomers, or amorphous solids of the compounds and compositions embodied herein.

As used herein, “pharmaceutically acceptable carrier” includes any and all solvents, dispersion media, coatings, surfactants, antioxidants, preservatives (e.g., antibacterial agents, antifungal agents), isotonic agents, absorption delaying agents, salts, preservatives, drugs, drug stabilizers, gels, binders, excipients, disintegration agents, lubricants, sweetening agents, flavoring agents, dyes, such like materials and combinations thereof, as would be known to one of ordinary skill in the art (see, for example, Remington's Pharmaceutical Sciences, 1990, incorporated herein by reference). Except insofar as any conventional carrier is incompatible with the active ingredient or method of use, its use in the pharmaceutical compositions is contemplated.

In some therapeutic embodiments, an “effective amount” of an agent is an amount that results in a reduction of at least one pathological parameter. Thus, for example, in some aspects of the present disclosure, an effective amount is an amount that is effective to achieve a reduction of at least about 10%, at least about 15%, at least about 20%, or at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, or at least about 95%, compared to the expected reduction in the parameter in an individual not treated with the agent.

An “effective amount” of a galectin-1-targeting compound may be an amount of the compound necessary to bring about a desired effect. For example, an effective amount of OTX-008 to inhibit growth or proliferation of human cancer cells, in vitro or in vivo, is an amount of compound required to observe inhibition of growth or proliferation of such cells, as compared to the growth or proliferation of such cells that are not contacted with OTX-008, but are otherwise identical to the cells contacted with OTX-008. It will be understood that “effective amount” therefore refers to low concentrations of compound which have a measurable but relatively small effect on the cells, as well as high concentrations of compound which have a measurable and substantial effect on the cells.

The term “therapeutically effective amount” is used herein, unless otherwise indicated, to describe an amount of a compound or composition which, in context, is used to produce or effect an intended therapeutic result. In an embodiment, the intended therapeutic result relates to the treatment of a hyperproliferative disease state, such as a tumor, including a carcinogenic tumor or other cancer or the treatment of a precancerous lesion or other cell(s) which express abnormal or foreign proteins or immunogens on a cell surface. With respect to an anticancer effect, that effect may be one or more of inhibiting further growth of tumor or cancer cells, inducing an antiangiogenic effect (e.g., by killing tumor endothelial cells), reducing the likelihood or eliminating metastasis or producing cell death in the tumor or cancer cells, resulting in a shrinkage of the tumor or a reduction in the number of cancer cells or preventing the regrowth of a tumor or cancer after the patient's tumor or cancer is in remission. As indicated, an anti-cancer agent may exhibit an anti-cancer effect alone and/or may enhance the ability of another anticancer agent to exhibit an anti-cancer effect. In an embodiment, the intended therapeutic result relates to the treatment of a hyperproliferative disease state that is a result or consequence of galectin-1 expression, function, or activity. In an embodiment, the intended therapeutic result is, at least in part, due to targeting of galectin-1 by a compound or composition. In an embodiment, targeting of galectin-1 involves physical interaction of galectin-1 and the compound or composition. In an embodiment, targeting of galectin-1 does not involve physical interaction of galectin-1 and the compound or composition, but nonetheless, involves a direct effect of the compound or composition on galectin-1, through one or more other molecules. In an embodiment, targeting of galectin-1 involves both direct physical interaction with galectin-1 as well as one or more indirect interactions with galectin-1.

In an embodiment, a galectin-1 targeting compound, such as, for example, OTX-008, binds to galectin-1 with a K_{d} in the range of about 1 μM to about 100 μM, about 5 μM to about 60 μM, about 10 μM to about 50 μM,

In an embodiment, a galectin-1 targeting compound, such as, for example, OTX-008, binds to galectin-1 with a K_{d} about 1 μM, about 2.5 μM, about 5 μM, about 7.5 μM, about 10 μM, about 15 μM, about 20 μM, about 25 μM, about 30 μM, about 35 μM, about 40 μM, about 45 μM, about 50 μM, about 55 μM, about 60 μM, about 65 μM, about 70 μM, about 75 μM, about 80 μM, about 85 μM, about 90 μM, about 95 μM, or about 100 μM.

In an embodiment, a galectin-1 targeting compound, such as, for example, OTX-008, binds to galectin-1 with a K_{d} of about at least 1 μM, about at least 2.5 μM, about at least 5 μM, about at least 7.5 μM, about at least 10 μM, about at least 15 μM, about at least 20 μM, about at least 25 μM, about at least 30 μM, about at least 35 μM, about at least 40 μM, about at least 45 μM, about at least 50 μM, about at least 55 μM, about at least 60 μM, about at least 65 μM, about at least 70 μM, about at least 75 μM, about at least 80 μM, about at least 85 about at least 90 μM, about at least 95 or about at least 100 μM.

In an embodiment, a galectin-1 targeting compound, such as, for example, OTX-008, binds to galectin-1 with a K_{d} of about less than 1 μM, about less than 2.5 μM, about less than 5 μM, about less than 7.5 μM, about less than 10 μM, about less than 15 μM, about less than 20 μM, about less than 25 μM, about less than 30 μM, about less than 35 μM, about less than 40 μM, about less than 45 μM, about less than 50 μM, about less than 55 μM, about less than 60 μM, about less than 65 μM, about less than 70 μM, about less than 75 μM, about less than 80 μM, about less than 85 μM, about less than 90 μM, about less than 95 μM, or about less than 100 μM.

In an embodiment, a therapeutically effective amount of a galectin-1 targeting compound, such as, for example, OTX-008, is an amount required to provide a concentration of about 30 μM. In an embodiment, a therapeutically effective amount of a galectin-1 targeting compound, such as, for example, OTX-008, is an amount required to provide a concentration of OTX-008 of a K_{d} value encompassed and/or disclosed herein.

In an embodiment, a galectin-1-targeting compound contacts one or more amino acid residues of galectin-1 by binding with at least one residue of galectin-1. In an embodiment, a galectin-1-targeting compound contacts one or more residues of galectin-1 from amino acid residues 6, 7, 8, 9, 10, 14, 15, 16, 17, 89, 90, 91, and/or 92 of human galectin-1 sequence set forth in SEQ ID NO:4.

In an embodiment, a galectin-1-targeting compound contacts one or more residues of galectin-1 corresponding to amino acid residues 6, 7, 8, 9, 10, 14, 15, 16, 17, 89, 90, 91, and/or 92 of the human galectin-1 sequence set forth in SEQ ID NO:4.

In an embodiment, a galectin-1-targeting compound contacts one or more amino acid residues of galectin-1 by binding with at least one residue of galectin-1, thereby attenuating the binding of lactose to the front face β-sheet. In an embodiment, OTX-008 contacts one or more amino acid residues of galectin-1 by binding with at least one residue of galectin-1, thereby attenuating the binding of lactose to the front face β-sheet. In an embodiment, the binding of OTX-008 is with the back face β-sheet of galectin-1.

In an embodiment, a galectin-1-targeting compound contacts one or more amino acid residues of galectin-1 by binding with at least one residue of galectin-1, thereby attenuating the binding of galectin-1 to one or more cell surface glycans. In some embodiments, the galectin-1 binding compound contacts one or more amino acid residues of galectin-1 by binding with at least one residue of galectin-1, thereby attenuating the binding of galectin-1 to one or more cell surface glycans. In an embodiment, the binding is with the back face β-sheet of galectin-1. In some embodiments, the galectin-1 binding compound is OTX-008.

The terms “coadministration” and “combination therapy” are used to describe a therapy in which at least two compounds are used to treat cancer or another disease state or condition, as described herein, at the same time. In an embodiment, the terms “coadministration” and “combination therapy” are used to describe a therapy in which at least two compounds are used to treat cancer or another disease state or condition as described herein, at the same time, wherein one of the compounds is a galectin-1-targeting compound. In an embodiment, the terms “coadministration” and “combination therapy” are used to describe a therapy in which at least two compounds are used to treat cancer or another disease state or condition as described herein at the same time, wherein one of the compounds is a galectin-1 binding compound such as, for example, OTX-008. In an embodiment, the activity of a galectin-1 binding compound and a co-administered composition is mediated, at least in part, via modulation of galectin-1, the other compound or compounds in the co-administered therapy may or may not act via modulation of galectin-1. In an embodiment, combination therapy with a galectin-1 binding compound is used to treat cancer or another disease state or condition, as described herein, at the same time. In another embodiment, a composition for combination therapy with a galectin-1 binding compound such as, for example, OTX-008, which composition comprises an effective amount for treatment, is used to treat cancer or another disease state or condition, as described herein, at the same time. In an embodiment, the result of coadministration with a galectin-1 binding compound such as, for example, OTX-008, may be additive of the treatment results obtained using each compound separately, either directly additive, or additive to a degree lesser than the results obtained with the two compounds separately. In an embodiment, the result of treatment via coadministration with a galectin-1 binding compound such as, for example, OTX-008, may be synergistic, to varying degrees. In an embodiment, the result of treatment via coadministration with a galectin-1 binding compound such as, for example, OTX-008, may be greater than the treatment results obtained using each compound separately. In an aspect, the result of treatment with a composition encompassed herein is such that, for one compound, the result of treatment is greater than that obtained with the compound separately, while the results of treatment with respect to the other compounds in the composition are about the same as the results of treatment obtained separately. In an aspect, the result of treatment for at least two compounds is greater than that obtained with the compounds separately, while the other compounds in the composition are about the same as the results of treatment obtained separately. In an aspect, the result of treatment for all compounds in the composition is greater than that obtained with the compounds separately. In an embodiment, the result of treatment via coadministration with a galectin-1 binding compound such as, for example, OTX-008, may be less than the treatment results obtained using each compound separately. In an aspect, the result of treatment with a composition encompassed herein is such that, for one compound, the result of treatment is less than that obtained with the compound separately, while the results of treatment with respect to the other compounds in the composition are about the same as the results of treatment obtained separately. In an aspect, the result of treatment for at least two compounds is less than that obtained with the compounds separately, while the other compounds in the composition are about the same as the results of treatment obtained separately. In an aspect, the result of treatment for all compounds in the composition is less than that obtained with the compounds separately.

Treatment can be prophylactic or therapeutic. In an embodiment, treatment can be initiated before, during, or after the development of the cancerous condition. As such, the phrases “inhibition of” or “effective to inhibit” may include prophylactic and/or therapeutic treatment, and may include prevention and/or reversal of the condition.

In some embodiments, although the term coadministration encompasses the administration of two active compounds to the patient at the same time, wherein the compounds are not administered to the patient at the same time, the effective amounts of the individual compounds will be present in the patient at the same time.

In an embodiment, a composition comprises a galectin-1-targeting compound with an optional carrier (e.g., a pharmaceutically acceptable carrier), which composition is added to cells in culture or used to treat a patient, such as a human. In an embodiment, a composition comprises OTX-008 with an optional carrier (e.g., a pharmaceutically acceptable carrier), which composition is added to cells in culture or used to treat a patient, such as a human. Where OTX-008 is used to treat a patient, it is preferably combined in a pharmaceutical composition with at least one pharmaceutically acceptable carrier, such as a larger molecule to promote stability, or with a pharmaceutically acceptable buffer that serves as a carrier.

OTX-008, or any other galectin-1-targeting compound, can be administered alone or in a pharmaceutically acceptable buffer, as an antigen in association with another protein, such as an immunostimulatory protein or with a protein carrier such as, but not limited to, keyhole limpet hemocyanin (KLH), bovine serum albumin (BSA), ovalbumin, or the like. It can also be used in adjuvant therapy, in combination with, for example, one or more chemotherapeutic agents like carboplatin or others known to one skilled in the art.

In an embodiment, a galectin-1-targeting compound, such as OTX-008, can be combined with a variety of physiological acceptable carriers for delivery to a patient including a variety of diluents or excipients known to those of ordinary skill in the art. For example, for parenteral administration, isotonic saline is preferred. For topical administration a cream, including a carrier such as dimethylsulfoxide (DMSO), or other agents typically found in topical creams that do not block or inhibit activity of the peptide, can be used. Other suitable carriers include, but are not limited to alcohol, phosphate buffered saline, and other balanced salt solutions.

In an embodiment, a composition comprising a galectin-1-targeting compound disclosed herein, such as OTX-008, is cytotoxic to a cell. In an embodiment, the cell is a cancer cell. In an embodiment, the galectin-1-targeting compound binds to the back face β-sheet of galectin-1. In an embodiment, the compound contacts one or more residues of galectin-1 corresponding to amino acid residues 6, 7, 8, 9, 10, 14, 15, 16, 17, 89, 90, 91, and/or 92 of human galectin-1 set forth in SEQ ID NO:4. In an embodiment, the galectin-1-targeting compound has one or more biological activities as described in detail elsewhere herein. In an embodiment, the measure of cytotoxicity is obtained by contacting the cell with a compound disclosed herein and observing the effects on the cell using the MTT assay, as described in detail elsewhere herein, and comparing the observed effects with the appropriate controls containing untreated cells that are otherwise identical to the compound-treated cells. Cell parameters providing a measure of cytotoxicity include, but are not limited to, cell proliferation as a function of galectin-1 mRNA expression and/or galectin-1 protein expression.

Also provided herein is a method of identifying a cell susceptible to treatment with a galectin-1-targeting compound disclosed herein, such as OTX-008. In an embodiment, a galectin-1-targeting compound is cytotoxic to a cell susceptible to treatment with a galectin-1-targeting compound. In an embodiment, the galectin-1-targeting compound binds to the back face β-sheet of galectin-1. In an embodiment, the galectin-1-targeting compound additionally has one or more of the biological activities as described herein.

In another embodiment, a galectin-1-targeting compound has an antiproliferative effect on such a cell. In another embodiment, galectin-1-targeting compound affects such a cell so that one or more metabolic or cell-cycle pathways in the cell are altered. In an embodiment, the galectin-1-targeting compound acts, at least in part, by binding to the back face β-sheet of galectin-1. In an embodiment, the compound acts by contacting one or more residues of galectin-1 corresponding to amino acid residues 6, 7, 8, 9, 10, 14, 15, 16, 17, 89, 90, 91, and/or 92 of human galectin-1 sequence set forth in SEQ ID NO:4. In an embodiment, a cell described herein is a cancer cell. In an embodiment, a cell expressing a lower than normal level of galectin-1 will be susceptible to treatment with a galectin-1-targeting compound. In an embodiment, a cell expressing a higher than normal level of an epithelial marker will be susceptible to treatment with a galectin-1-targeting compound. In an embodiment, a cell expressing a higher than normal level of E-cadherin will be susceptible to treatment with a galectin-1-targeting compound. In another embodiment, an epithelial cell expressing a higher than normal level of keratin-8 and/or keratin-18 will be susceptible to treatment with a galectin-1-targeting compound. In an embodiment, a cell expressing a low level of a mesenchymal marker will be susceptible to treatment with a galectin-1-targeting compound. In an embodiment, a cell expressing a low level of vimentin will be susceptible to treatment with a galectin-1-targeting compound. In another embodiment, a cell having two or more of the characteristics described herein will be susceptible to treatment with a galectin-1-targeting compound. In an aspect, a method of predicting whether a cell will be susceptible to treatment with a galectin-1-targeting compound disclosed herein, such as OTX-008, comprises contacting the cell with a composition comprising a galectin-1-targeting compound. In another aspect, the composition is a pharmaceutical composition.

In an embodiment, a composition comprising OTX-008 is anti-angiogenic, via modulation of galectin-1. Angiogenesis is involved in numerous biological functions in the body, from normal processes like embryogenesis and wound healing to abnormal processes like tumor growth, arthritis, restenosis, atherosclerosis, diabetic retinopathy, neovascular glaucoma, and endometriosis. The compositions and methods herein are useful for treating processes and/or conditions in which galectin-1 plays a role. In an embodiment, the anti-angiogenic activity of the galectin-1-targeting compound occurs, at least in part, by binding to the back face β-sheet of galectin-1.

In an embodiment, encompassed herein is a method for inhibiting endothelial cell proliferation in a patient (e.g., a mammal such as a human), via modulation of galectin-1. In an aspect, this encompasses administering to a patient an amount of a composition (typically a pharmaceutical composition) effective to inhibit the growth of endothelial cells, wherein the composition comprises a galectin-1 targeting compound, such as, for example, OTX-008. In an embodiment, encompassed herein is a method for inhibiting endothelial cell proliferation in vitro (e.g., in a cell culture). In an aspect, this encompasses contacting cells with an amount of a composition effective to prevent and/or reduce the growth of endothelial cells, wherein the composition comprises OTX-008. In an embodiment, the activity of inhibiting endothelial cell proliferation of the galectin-1-targeting compound takes place, at least in part, by galectin-1-targeting compound binding to the back face β-sheet of galectin-1. In an embodiment, the compound acts by contacting one or more residues of galectin-1 corresponding to amino acid residues 6, 7, 8, 9, 10, 14, 15, 16, 17, 89, 90, 91, and/or 92 of human galectin-1 (SEQ ID NO:4).

For determining the amount of endothelial cell proliferation in vivo, various methods known to one of skill in the art could be used, as demonstrated herein. For example, for evaluation of endothelial cell growth in tumors, tissue sections can be appropriately stained to quantify vessel density. For determining the amount of endothelial cell proliferation in vitro, an Endothelial Cell Proliferation Assay can be used that involves the uptake of tritiated thymidine by cells in cell culture. In an embodiment, OTX-008 that is “active” for inhibiting endothelial cell proliferation is one that causes an at least 10% reduction in endothelial cell proliferation at an OTX-008 concentration lower than 10⁻⁴ M. Alternatively, inhibition of endothelial cell proliferation for an “active” OTX-008 in vitro is preferably at an IC50 level of less than 80 μM (more preferably less than 50 μM, and even more preferably less than 25 μM). In an embodiment, encompassed herein is a method for determining the amount of endothelial cell proliferation in vitro, wherein the method comprises an endothelial cell proliferation assay. In an embodiment, a measurement of inhibition of endothelial cell proliferation as described herein can be used to identify a therapeutically effective amount of a compound, such as, but not limited to, OTX-008. In an embodiment, encompassed herein is a method of correlating a therapeutically effective amount of a compound, as described elsewhere herein, with the amount of a compound considered to be active in the in vitro assays described herein.

In an embodiment, encompassed herein is a method for inhibiting angiogenic-factor mediated intercellular adhesion molecule (ICAM) expression down-regulation (and/or promoting ICAM expression) in a patient, via modulation of galectin-1. In an embodiment, the anti-angiogenic activity of the galectin-1-targeting compound occurs, at least in part, by galectin-1-targeting compound binding to the back face β-sheet of galectin-1. In an embodiment, the compound acts by contacting one or more residues of galectin-1 corresponding to amino acid residues 6, 7, 8, 9, 10, 14, 15, 16, 17, 89, 90, 91, and/or 92 of human galectin-1 sequence set forth in SEQ ID NO:4. In an embodiment, this involves administering to a patient an amount of a composition effective to prevent and/or reduce the amount of ICAM expression down-regulation, wherein the composition comprises galectin-1 targeting compound, such as, for example, OTX-008, and functions via modulation of galectin-1. In an embodiment, the amount of a compound, whether alone or as part of a composition, effective to prevent and/or reduce the amount of ICAM expression down-regulation is a therapeutically-effective amount, as described elsewhere herein. Analogously, the present invention provides a method for inhibiting angiogenic-factor mediated inter-cellular adhesion molecule expression down-regulation (and/or promoting ICAM expression) in vitro (e.g., in a cell culture). This method involves contacting cells with an amount of a composition effective to prevent and/or reduce the amount of ICAM expression down-regulation, wherein the composition comprises a galectin-1 targeting compound, such as, for example, OTX-008. In an embodiment, expression of ICAM, such as ICAM-1 protein, can be examined by flow cytometry with R-phycoerythrin-conjugated anti-ICAM-1 (CD54) monoclonal antibody or mouse IgG1 isotype control. In another embodiment, expression of ICAM nucleic acid can be monitored for up- or down-regulation. It will be understood by the skilled artisan, when armed with the disclosure set forth herein, that the nucleic acid expression patterns may differ from protein levels of ICAM expressed, even when based on the same sample and measured at the same time. The present disclosure provides the skilled artisan with the guidance necessary to correlate in vitro results with the results obtained upon treatment of a mammal with a compound or composition set forth herein, so that a therapeutically effective amount of a compound or composition can be correlated with the in vitro assay using the compound or composition.

Galectin-1 participates in the resistance of cancer cells to chemotherapy and radiotherapy. In an embodiment, a method is provided for reducing this resistance and increasing the sensitivity of a cancer to chemotherapy and/or radiotherapy treatment in a patient by the administration of an effective amount of a galetin-1 targeting compound, such as, for example, OTX-008.

Galectin-1 plays a role in the tumor immune escape process. In an embodiment, a method is provided for reducing tumor immune escape in a patient by the administration of an effective amount of a galetin-1 targeting compound, such as, for example, OTX-008.

In an embodiment, a method is provided for increasing the infiltration of leukocytes into tumor tissue in a patient (e.g., a mammal such as a human). In an embodiment, this involves administering to a patient an amount of a composition effective to increase the amount of white blood cells (leukocytes) that can infiltrate into the tumor tissue through blood vessels via modulation of galectin-1, wherein the composition comprises a galecting-1 targeting compound, such as, for example, OTX-008. In an embodiment, the leukocyte stimulating activity of a galectin-1-targeting compound occurs, at least in part, by galectin-1-targeting compound binding to the back face β-sheet of galectin-1. In an embodiment, the compound acts by contacting one or more residues of galectin-1 corresponding to amino acid residues 6, 7, 8, 9, 10, 14, 15, 16, 17, 89, 90, 91, and/or 92 of the human galectin-1 sequence set forth in SEQ ID NO:4.

In an embodiment, a method is provided for inhibiting angiogenesis (i.e., new blood vessel formation) in a patient (e.g., a mammal, such as a human). In an embodiment, this involves administering to a patient an amount of a composition effective to prevent and/or reduce angiogenesis, via galectin-1 modulation, wherein the composition comprises OTX-008. In another embodiment, a method is provided for inhibiting angiogenesis in vitro (e.g., in a cell culture). This method involves contacting cells with an amount of a composition effective to prevent and/or reduce angiogenesis via modulation of galectin-1, wherein the composition comprises OTX-008. In an embodiment, the angiogenesis-inhibiting activity of the galectin-1-targeting compound occurs, at least in part, by galectin-1-targeting compound binding to the back face β-sheet of galectin-1. In an embodiment, the compound acts by contacting one or more residues of galectin-1 corresponding to amino acid residues 6, 7, 8, 9, 10, 14, 15, 16, 17, 89, 90, 91, and/or 92 of the human galectin-1 sequence set forth in SEQ ID NO:4. For determining the amount of angiogenesis in vivo, various methods known to one of skill in the art could be used. For example, for evaluation of angiogenesis in tumors, tissue sections can be appropriately stained to quantify vessel density. For determining the amount of angiogenesis in vitro, an angiogenesis assay can be used that involves the disappearance of endothelial cell sprouting in cell culture. In an embodiment, a compound that is “active” for angiogenesis inhibition is preferably one that causes an at least 10% reduction in endothelial cell sprouting at a concentration lower than 10⁻⁴ M. In another embodiment, inhibition of angiogenesis for a composition comprising OTX-008 in vitro is preferably at a level of less than 85% sprouting (more preferably less than 75% sprouting, even more preferably 50% sprouting, and even more preferably less than 35%) as determined using a collagen gel-based assay as known in the art, such as that described in detail in U.S. Patent Publication No. 2008/0300164 A1 (Ser. No. 11/664,641), which is incorporated by reference herein in its entirety.

In an embodiment, a method is provided for inhibiting tumorigenesis in a patient via modulation of galectin-1. In an embodiment, the method comprises administering to a patient an amount of a composition effective to prevent and/or reduce tumor growth, via modulation of galectin-1, wherein the composition comprises OTX-008. In an embodiment, the activity of inhibiting tumorigenesis by the galectin-1-targeting compound occurs, at least in part, by galectin-1-targeting compound binding to the back face β-sheet of galectin-1. In an embodiment, the compound acts by contacting one or more residues of galectin-1 corresponding to amino acid residues 6, 7, 8, 9, 10, 14, 15, 16, 17, 89, 90, 91, and/or 92 of human galectin-1 sequence set forth in SEQ ID NO:4. Methods of determining the inhibition of tumorigenesis are well known to those of skill in the art, including evaluation of tumor shrinkage, survival, etc.

In an embodiment a method is provided for treatment of cancer in a patient, wherein the treatment of the cancer includes preventing or slowing the growth of the cancer. In an embodiment a method is provided for treatment of cancer in a patient, wherein the treatment of the cancer includes preventing the spread of a tumor associated with the cancer. In an embodiment a method is provided for treatment of cancer in a patient, wherein the treatment of the cancer includes preventing the spread of one or more metastases associated with the cancer. In an embodiment a method is provided for treatment of cancer in a patient, wherein the treatment of the cancer includes reducing the size of a tumor associated with the cancer. In an embodiment a method is provided for treatment of cancer in a patient, wherein the treatment of the cancer includes preventing the recurrence of cancer treated previously. In an embodiment, the anti-cancer activity of the galectin-1-targeting compound occurs, at least in part, by galectin-1-targeting compound binding to the back face β-sheet of galectin-1. In an embodiment, the compound acts by contacting one or more residues of galectin-1 corresponding to amino acid residues 6, 7, 8, 9, 10, 14, 15, 16, 17, 89, 90, 91, and/or 92 of human galectin-1 sequence set forth in SEQ ID NO:4.

As will be understood based on the disclosure set forth herein, cancers in which galectin-1 is believed to play a role are cancers that may be treated using the compositions and methods set forth herein. Such cancers include, but are not limited to, ovarian cancer, squamous cell carcinoma, cancers of the digestive system, including stomach, liver, and colon cancer, cancers of the thyroid, cancers of the endometrium and ovaries, including adenocarcinoma of the endometrium, uterine cancers, including uterine adenocarcinomas and uterine smooth muscle tumors, breast cancer, prostate cancer, bladder cancer, head and neck cancers, including gliomas, kidney cancers, pancreatic cancer, including pancreatic ductal adenocarcinoma, nonsmall-cell lung cancer, and melanoma.

Cancers to be treated may include, but are not limited to, melanoma, basal cell carcinoma, colorectal cancer, pancreatic cancer, breast cancer, prostate cancer, lung cancer (including small-cell lung carcinoma and non-small-cell lung carcinoma, leukemia, lymphoma, sarcoma, ovarian cancer, Kaposi's sarcoma, Hodgkin's lymphoma, Non-Hodgkin's lymphoma, multiple myeloma, neuroblastoma, rhabdomyosarcoma, primary thrombocytosis, primary macroglobulinemia, small-cell lung tumors, primary brain tumors, stomach cancer, head and neck cancers, malignant pancreatic insulanoma, malignant carcinoid, urinary bladder cancer, premalignant skin lesions, testicular cancer, lymphomas, thyroid cancer, neuroblastoma, esophageal cancer, genitourinary tract cancer, malignant hypercalcemia, cervical cancer, kidney cancer, endometrial cancer, glioblastoma, and adrenal cortical cancer.

In some embodiments, the cancer is a carcinoma, a sarcoma, a blood borne hematologic cancer, or a germ line cancer. In some embodiments, the cancer is a breast cancer, ovarian cancer, melanoma, colon cancer, lung cancer, or a squamous cell carcinoma.

In some aspects, the cancer is a primary cancer. In some aspects, the cancer is metastatic. In some aspects, the cancer is a tumor associated cancer. In some embodiments, the cancer comprises galetin-1 positive cells. In some embodiments, the cancer comprises galetin-1 negative cells. In other aspects, the cancer is a blood borne cancer. Such hematological blood borne malignancies include, for example, leukemia, lymphoma, multiple myeloma, acute myelogenous leukemia, myelodysplastic syndrome, non-Hodgkins lymphoma, or follicular lymphoma.

In some aspects, a cancer may be of epithelial cell origin (carcinoma), non-hematopoietic mesenchymal cell origin (sarcoma), hematopoietic cell origin, germ cell origin, or a cancer whose origin or developmental lineages is unknown. Examples of carcinomas of epithelial origin that may be treated include, but are not limited to, adenocarcinoma, squamous cell carcinoma, adenosquamous carcinoma, anaplastic carcinoma, large cell carcinoma, small cell carcinoma, ovarian cancer, melanoma, and cancer of the lung, breast, prostate, colon, rectum, and/or pancreas, and metastasis thereof. Examples of sarcomas of mesenchymal cell origin include, but are not limited to, osteosarcoma, chondrosarcoma, leiomyosarcoma, rhabdomyosarcoma, fibrosarcoma, schwannoma, fibroblastic osteosarcoma, myofibroblastic sarcoma, malignant fibrous histiocytoma, fibromyxosarcoma, spindle cell sarcoma, and adamantinoma.

In some aspects, a cancer may demonstrate an epithelial to mesenchymal transition phenotype (EMT). Epithelial-mesenchymal transition or transformation (EMT) is defined by the loss of epithelial phenotype and the acquisition of a mesenchymal phenotype. The EMT is an orchestrated series of events in which cell-cell and cell-extracellular matrix interactions are altered to release epithelial cells from the surrounding tissue, the cytoskeleton is reorganized to confer the ability to move through a three-dimensional extracellular matrix, and a new transcriptional program is induced to maintain the mesenchymal phenotype. It is characterized by, for example, the loss of cell adhesion, cadherin switching (down-regulation of E-cadherin and up-regulation of mesenchymal cadherins such as N-cadherin or cadherin-11), and increased cell mobility. While EMT is essential in embryonic development, it is potentially destructive when deregulated, and it is becoming increasingly understood that inappropriate utilization of EMT mechanisms is an integral component of the progression of many tumors of epithelial tissues. See, for example, Radisky, 2005, J Cell Sci; 118:4325-4326.

In an embodiment, a method is provided for treatment of a cancer in a patient, wherein the cancer is associated with overexpression of galectin-1. As will be understood by the skilled artisan, overexpression of galectin-1 encompasses levels of expression of galectin-1 exceeding typical, normal, or “healthy” levels of galectin-1 expression. In an embodiment, the method comprises administering to a patient a therapeutically effective amount of a galectin-1-targeting composition effective to treat the cancer in a patient, wherein the cancer is associated with overexpression of galectin-1. In an embodiment, the method comprises administering to a patient a therapeutically effective amount of OTX-008 effective to treat the cancer in a patient, wherein the cancer is associated with overexpression of galectin-1. In an embodiment, the cancer cells overexpress galectin-1. In another embodiment, the cancer cells do not overexpress galectin-1, but at least one non-cancerous cell in the patient overexpresses galectin-1, wherein the non-cancerous cell is responsible, at least in part, for the cancer. In another embodiment, the cancer cells overexpress galectin-1, and at least one non-cancerous cell in the patient overexpresses galectin-1, wherein the non-cancerous cell is responsible, at least in part, for the cancer. In an aspect of an embodiment, a galectin-1-targeting composition, such as a composition comprising OTX-008, when administered to a patient, contacts at least one cancer cell in the patient. In another aspect of an embodiment, a galectin-1-targeting composition, such as a composition comprising OTX-008, when administered to a patient, contacts at least one cancer cell in the patient, wherein the cancer cell overexpresses galectin-1.

Compositions useful for treatment as described herein include pharmaceutically acceptable salts of the compounds in the composition, including OTX-008. As used herein, the term pharmaceutically acceptable salts or complexes refers to salts or complexes that retain the desired biological activity of the parent compound and exhibit minimal, if any, undesired toxicological effects. While it will be understood that a galectin-1 targeting compound such as OTX-008 may practically form different useful salts than a small molecule drug, nonlimiting examples of such salts are (a) acid addition salts formed with inorganic acids (for example, hydrochloric acid, hydrobromic acid, sulfuric acid, phosphoric acid, nitric acid, and the like), and salts formed with organic acids such as acetic acid, oxalic acid, tartaric acid, succinic acid, malic acid, ascorbic acid, benzoic acid, tannic acid, pamoic acid, alginic acid, polyglutamic acid, naphthalenesulfonic acids, naphthalenedisulfonic acids, and polygalacturonic acid; (b) base addition salts formed with polyvalent metal cations such as zinc, calcium, bismuth, barium, magnesium, aluminum, copper, cobalt, nickel, cadmium, sodium, potassium, and the like, or with an organic cation formed from N,N-dibenzylethylene-diamine, ammonium, or ethylenediamine; or (c) combinations of (a) and (b); e.g., a zinc tannate salt or the like.

Modifications of a compound can affect the solubility, bioavailability and rate of metabolism of the active species, thus providing control over the delivery of the active species. Further, the modifications can affect the anticancer activity of the compound, in some cases increasing the activity over the parent compound. This can easily be assessed by preparing the derivative and testing its anticancer activity according to the methods encompassed herein, or other methods known to those skilled in the art.

The agents of the present disclosure can be administered to a subject by any suitable means including, but not limited to, for example, oral, rectal, nasal, topical (including transdermal, aerosol, buccal and/or sublingual), vaginal, parenteral, intradermal, intravesical, intra joint, intra-arteriole, intraventricular, intracranial, intraperitoneal, intranasal, by inhalation, intralesional (for example, by injection into or around a tumor), or by implanted reservoir. “Parenteral” as used herein includes intradermal, subcutaneous, intravenous, intramuscular, intra-articular, intra-synovial, infrasternal, intrathecal, intrahepatic, intralesional, intracranial, and/or infusion techniques.

For parenteral administration in an aqueous solution, for example, the solution should be suitably buffered if necessary and the liquid diluent first rendered isotonic with sufficient saline or glucose. These particular aqueous solutions are especially suitable for intravenous, intramuscular, subcutaneous, intraperitoneal, and intratumoral administration. In this connection, sterile aqueous media that can be employed will be known to those of skill in the art. Some variation in dosage will necessarily occur depending on the condition of the subject being treated. The person responsible for administration will, in any event, determine the appropriate dose for the individual subject. Moreover, for human administration, preparations should meet sterility, pyrogenicity, and general safety and purity standards as required by the FDA. Such preparation may be pyrogen-free.

For enteral administration, the inhibitor may be administered in a tablet or capsule, which may be enteric coated, or in a formulation for controlled or sustained release. Many suitable formulations are known, including polymeric or protein microparticles encapsulating drug to be released, ointments, gels, or solutions which can be used topically or locally to administer drug, and even patches, which provide controlled release over a prolonged period of time. These can also take the form of implants. Such an implant may be implanted within the tumor.

The compounds of the present invention can also be provided in a lyophilized form. Such compositions may include a buffer, e.g., bicarbonate, for reconstitution prior to administration, or the buffer may be included in the lyophilized composition for reconstitution with, e.g., water. The lyophilized composition may further comprise a suitable vasoconstrictor, e.g., epinephrine. The lyophilized composition can be provided in a syringe, optionally packaged in combination with the buffer for reconstitution, such that the reconstituted composition can be immediately administered to a patient.

In an embodiment, a composition is formulated to independently include between about 0.1 milligram and about 2000 milligrams of each active compound, including anti-cancer compounds, anti-angiogenic compounds, and anti-emetic compounds, among others, as well as bioactive compounds. In an embodiment, a composition is formulated to independently include any value between about 0.1 milligram and about 2000 milligrams of each active compound, including anti-cancer compounds, anti-angiogenic compounds, and anti-emetic compounds, among others, as well as bioactive compounds. In an embodiment, a composition is formulated to independently include any value between about 0.1 milligram and about 2000 milligrams of each active compound per unit dosage form, including anti-cancer compounds, anti-angiogenic compounds, and anti-emetic compounds, among others, as well as bioactive compounds. It will be understood that the solubility limitations of any compound, including OTX-008, will be taken into consideration when developing dosage forms and dosing regimens.

In an embodiment, compositions comprising a galectin-1-targeting compound, such as OTX-008, can be administered as a single dose or in multiple doses. Preferably the dose is an effective amount as determine by the standard methods described herein and includes about 1 microgram to about 1,000 micrograms pretreatment, about 25 micrograms to about 500 micrograms pretreatment, about 50 to about 250 micrograms pretreatment, about 2 micrograms to about 100 micrograms pretreatment, about 5 micrograms to about 75 micrograms pretreatment, about 10 micrograms to about 50 micrograms pretreatment, about 20 micrograms to about 40 micrograms pretreatment, and as discussed in greater detail elsewhere herein. Those skilled in the art of clinical trials will be able to optimize dosages of OTX-008-containing compositions through standard trial studies, in view of the disclosure encompassed herein.

In an embodiment, OTX-008, and the therapeutic composition as encompassed herein, is included in a pharmaceutically acceptable carrier or diluent in an amount sufficient to deliver to a patient a therapeutically effective amount for the desired indication, without causing serious toxic effects in the patient treated. In an embodiment, OTX-008 is administered to a patient in the range of about 1 mg/day to about 2000 mg/day. In another embodiment, OTX-008 is administered to a patient in the range of about 5 mg/day to about 1500 mg/day, about 10 mg/day to about 1250 mg/day, about 20 mg/day to about 1000 mg/day, about 30 mg/day to about 750 mg/day, about 40 mg/day to about 500 mg/day, about 50 mg/day to about 250 mg/day, and about 60 mg/day to about 100 mg/day.

In some embodiments, OTX-008 is administered to a patient in the range of about 500 mg/day to about 600 mg/day. In an embodiment, OTX-008 is administered to a patient in a dose of about 5 mg/day, about 10 mg/day, about 15 mg/day, about 20 mg/day, about 25 mg/day, about 30 mg/day, about 35 mg/day, about 40 mg/day, about 45 mg/day, about 50 mg/day, about 65 mg/day, about 55 mg/day, about 60 mg/day, about 65 mg/day, about 70 g/day, about 75 mg/day, about 80 mg/day, about 85 mg/day, about 90 mg/day, about 95 mg/day, or about 100 mg/day.

In another embodiment, a dose of the active compound for all of the herein-mentioned conditions is in the range from about 10 ng/kg to about 300 mg per kilogram body weight of the recipient/patient per day, from about 0.1 mg/kg to about 100 mg/kg per day, from about 0.5 mg/kg to about 25 mg/kg per day. The compound is conveniently administered in any suitable unit dosage form.

Unless otherwise specified to the contrary, the methods encompassed herein should be understood to embody the use of a compound encompassed herein, a composition comprising a compound encompassed herein, or a composition comprising at least two compounds disclosed herein. As will be understood from the present disclosure, a composition comprising one or more compounds may be a pharmaceutically-acceptable composition, or other composition including at least one substance in addition to the compound.

It will be understood, based on the disclosure set forth herein, in view of the skill in the art, that specific dosage for compounds and compositions encompassed herein may be determined empirically through clinical and/or pharmacokinetic experimentation and that such dosages may be adjusted according to prespecified toxicity criteria. It will also be understood that a specific dosage and treatment regimen for any particular patient will depend upon a variety of factors, including the activity of the specific compound employed, the age, body weight, general health, sex, diet, time of administration, rate of excretion, drug combination, and the judgment of the treating physician and the severity of the particular disease or condition being treated.

Compounds and compositions encompassed herein, or derivatives of the same, can be prepared according to the methods encompassed herein and/or known the art.

In an embodiment, the compositions encompassed herein are administered by way of a single dosage form. In another embodiment, the compositions are administered in multiple dosage forms. In an aspect, different agents are administered in different dosage forms.

In an embodiment, a method for treating cancer comprises administering to a mammal in need thereof a therapeutically effective amount of a composition comprising at least two anti-cancer agents, or pharmaceutically acceptable salts, hydrates, solvates, stereoisomers, or amorphous solids thereof, wherein one of the compounds is OTX-008, and the composition acts, at least in part, via modulation of galectin-1. In an embodiment, slowing or ceasing tumor progression is a sign of treatment of cancer (e.g., in advanced and/or metastatic cancer).

In an embodiment, disclosed herein are methods of treating a subject that can benefit from administration of a galectin-1-targeting compound. In an aspect, the subject is a human patient. In an embodiment, a method of treating cancer in a patient having cancer includes contacting one or more cancerous cells in the subject with a therapeutically effective amount of a galectin-1 targeting compound. In an embodiment, the compound is OTX-008. In an embodiment, a method of inhibiting growth and/or proliferation of a cancer cell includes contacting a cancer cell with a therapeutically effective amount of a galectin-1 targeting compound. In an embodiment, the compound is OTX-008. In an embodiment, a method of inhibiting growth and/or proliferation of a cancer cell includes contacting a cancer cell with a therapeutically effective amount of a galectin-1 targeting compound, wherein the galectin-1-targeting compound has a biological activity as described in detail elsewhere herein. In an embodiment, the biological activity comprises binding of the galectin-1-targeting compound to the back face β-sheet of galectin-1. In an embodiment, the compound acts by contacting one or more residues of galectin-1 selected from the group consisting of residues 6, 7, 8, 9, 10, 14, 15, 16, 17, 89, 90, 91, and 92 of human galectin-1 sequence set forth in SEQ ID NO:4. In an embodiment, a method of inhibiting tumor growth and/or metastasis in a patient having a tumor includes contacting a tumor with a therapeutically effective amount of a galectin-1 targeting compound. In an embodiment, the compound is OTX-008. In an embodiment, a method of inhibiting galectin-1 activity and/or expression includes contacting a cell with a therapeutically effective amount of a galectin-1 targeting compound. In an embodiment, the compound is OTX-008.

In an embodiment, a method of treating a patient includes administration of a composition encompassed herein to treat cancer, and the treatment of the cancer includes at least one of preventing or slowing the growth of the cancer, preventing the spread of a tumor associated with the cancer, preventing the spread of one or more metastases associated with the cancer, reducing the size of a tumor associated with the cancer, and preventing the recurrence of cancer which was treated previously, wherein galectin-1 plays a role in the progression of the cancer. In an aspect, an administered composition comprises OTX-008, and the composition acts, at least in part, via modulation of galectin-1.

A subject suffering from cancer can be treated by administering to the subject an effective amount of a composition comprising a galectin-1 targeting compound, as described herein, optionally in a pharmaceutically acceptable carrier or diluent, either alone, or in combination with other known anticancer or pharmaceutical agents. This treatment can also be administered in conjunction with other conventional cancer therapies, such as radiation treatment, chemotherapy, or surgery.

In some aspects of the methods of the present invention, a method further includes the administration of one or more additional therapeutic agents. One or more additional therapeutic agents may be administered before, after, and/or coincident to the administration of a galectin-1 targeting compound described herein. A galectin-1 targeting compound as described herein and additional therapeutic agents may be administered separately or as part of a mixture or cocktail. In some aspects of the present invention, the administration of a galectin-1 targeting compound may allow for the effectiveness of a lower dosage of other therapeutic modalities when compared to the administration of the other therapeutic modalities alone, providing relief from the toxicity observed with the administration of higher doses of the other modalities.

Additional therapeutic treatments include, but are not limited to, surgical resection, radiation therapy, hormone therapy, vaccines, antibody based therapies, whole body irradiation, bone marrow transplantation, peripheral blood stem cell transplantation, the administration of chemotherapeutic agents (also referred to herein as “antineoplastic chemotherapy agent,” “antineoplastic agents,” or “antineoplastic chemotherapeutic agents”), cytokines, antiviral agents, immune enhancers, tyrosine kinase inhibitors, signal transduction inhibitors, antibiotics, antimicrobial agents, a TLR agonist (such as for example, bacterial lipopolysaccharides (LPS) or a CpG oligonucleotide (ODN)), an inhibitor of IDO, such as, for example, 1-MT, and adjuvants.

A chemotherapeutic agent may be, for example, a cytotoxic chemotherapy agent, such as, for example, epidophyllotoxin, procarbazine, mitoxantrone, platinum coordination complexes such as cisplatin and carboplatin, leucovorin, tegafur, paclitaxel, docetaxol, vincristine, vinblastine, methotrexate, cyclophosphamide, gemcitabine, estramustine, carmustine, adriamycin (doxorubicin), etoposide, arsenic trioxide, irinotecan, epothilone derivatives, navelbene, CPT-11, anastrazole, tetrazole, capecitabine, reloxafine, ifosamide, and droloxafine.

A chemotherapeutic agent may be, for example, an alkylating agent, such as, for example, irofulven, nitrogen mustards (such as chlorambucil, cyclophosphamide, ifosfamide, mechlorethamine, melphalan, and uracil mustard), aziridines (such as thiotepa), methanesulphonate esters (such as busulfan), nitroso ureas (such as carmustine, lomustine, and streptozocin), platinum complexes (such as cisplatin and carboplatin), and bioreductive alkylators (such as mitomycin, procarbazine, dacarbazine and altretamine), ethylenimine derivatives, alkyl sulfonates, triazenes, pipobroman, temozolomide, triethylene-melamine, and triethylenethiophosphoramine.

A chemotherapeutic agent may be an antimetabolite, such as, for example, a folate antagonist (such as methotrexate and trimetrexate), a pyrimidine antagonist (such as fluorouracil, fluorodeoxyuridine, CB3717, azacitidine, cytarabine, gemcitabine, and floxuridine), a purine antagonist (such as mercaptopurine, 6-thioguanine, fludarabine, and pentostatin), a ribonucleotide reductase inhibitor (such as hydroxyurea), and an adenosine deaminase inhibitor.

A chemotherapeutic agent may be a DNA strand-breakage agent (such as, for example, bleomycin), a topoisomerase II inhibitor (such as, for example, amsacrine, dactinomycin, daunorubicin, idarubicin, mitoxantrone, doxorubicin, etoposide, and teniposide), a DNA minor groove binding agent (such as, for example, plicamydin), a tubulin interactive agent (such as, for example, vincristine, vinblastine, and paclitaxel), a hormonal agent (such as, for example, estrogens, conjugated estrogens, ethinyl estradiol, diethylstilbesterol, chlortrianisen, idenestrol, progestins (such as hydroxyprogesterone caproate, medroxyprogesterone, and megestrol), and androgens (such as testosterone, testosterone propionate, fluoxymesterone, and methyltestosterone)), an adrenal corticosteroid (such as, for example, prednisone, dexamethasone, methylprednisolone, and prednisolone), a leutinizing hormone releasing agent or gonadotropin-releasing hormone antagonist (such as, for example, leuprolide acetate and goserelin acetate), an antihormonal agent (such as, for example, tamoxifen), an antiandrogen agent (such as flutamide), an antiadrenal agent (such as mitotane and aminoglutethimide), and a natural product or derivative thereof (such as, for example, vinca alkaloids, antibiotics, enzymes and epipodophyllotoxins, including, for example vinblastine, vincristine, vindesine, bleomycin, dactinomycin, daunorubicin, doxorubicin, epirubicin, idarubicin, ara-C, paclitaxel, mithramycin, deoxyco-formycin, mitomycin-C, L-asparaginase, and teniposide.

A chemotherapeutic agent may be an anti-angiogenic agent, such as, for example, anginex, compound 1097, bridged compound 4, JIL31, JIL50, JIL54, JIL70, sunitinib (Sutent®, Pfizer), sorafenib (Nexavar®, Bayer) and bevacizumab (Avastin®, Genentech).

In some aspects of the methods of the present invention, at least one additional therapeutic agent includes radiation therapy. In some aspects, radiation therapy includes localized radiation therapy delivered to the tumor. In some aspects, radiation therapy includes total body irradiation.

Cytokines include, but are not limited to, IL-1 α, IL-1 β, IL-2, IL-3, IL-4, IL-6, IL-8, IL-9, IL-10, IL-12, IL-13, IL-15, IL-18, IL-19, IL-20, IFN-α, IFN-β, IFN-γ, tumor necrosis factor (TNF), transforming growth factor-β(TGF-β), granulocyte colony stimulating factor (G-CSF), macrophage colony stimulating factor (M-CSF), granulocyte-macrophage colony stimulating factor (GM-CSF), and or Flt-3 ligand. Antibody therapeutics, include, for example, trastuzumab (Herceptin) and antibodies to cytokines, such as IL-10 and TGF-β.

In some aspects of the methods of the present invention, the administration of a galectin-1 targeting compound as described herein and the at least one additional therapeutic agent demonstrate therapeutic synergy. In some aspects of the methods of the present invention, a measurement of response to treatment observed after administering both a galectin-1 targeting compound and the additional therapeutic agent is improved over the same measurement of response to treatment observed after administering either the galectin-1 targeting compound or the additional therapeutic agent alone.

In some embodiments, a therapeutic composition is administered on a schedule once a day. In some embodiments, a therapeutic composition is administered more than once a day, for example, twice a day, three times a day, four times a day, five times a day, or more. In some embodiments, a therapeutic composition is administered less frequently than once a day, for example, administered once every two days, once every three days, once every four days, once every five days, once every six days, or once every seven days. In some embodiments, a therapeutic composition is administered less frequently than once a week. In some embodiments, a therapeutic composition is administered once a month. In some embodiments, a therapeutic composition is administered twice a month.

In an aspect, based on the disclosure set forth herein, the skilled artisan will understand how to modify the compounds included in the composition, how to add or remove specific compounds from the composition, and how to adjust the dosage amounts, the dosage frequency, and the route of administration in order to optimize the treatment for a specific subject having a specific type of cancer or cancers. The concentration of active compounds in the composition will depend on absorption, distribution, inactivation, and excretion rates of the compound, as well as other factors known to those of skill in the art. It is to be noted that dosage values will also vary with the severity of the condition to be alleviated. It is to be further understood that for any particular subject, specific dosage regimens should be adjusted over time according to the individual need and the professional judgment of the individual administering or supervising the administration of the compositions, and that the concentration ranges set forth herein are exemplary only and are not intended to limit the scope or practice of the claimed composition. The active ingredient may be administered at once, or may be divided into a number of smaller doses to be administered at varying intervals of time.

The findings of the present disclosure can be used in methods that include, but are not limited to, methods for treating cancer, methods for inhibiting cell growth, and methods for killing cells. With the methods described herein, contacting steps may occur in vitro. In some embodiments, the contacting step occurs in vivo. In some embodiments, the cells are present in a cell culture, a tissue, an organ, or an organism. In some embodiments, the cells are mammalian cells. In some embodiments, the cells include cancer cells. In some embodiments, the cells include non-cancerous cells

As used herein, the term “subject” includes, but is not limited to, humans and non-human vertebrates. In preferred embodiments, a subject is a mammal, particularly a human. A subject may be an “individual,” “patient,” or “host. Non-human vertebrates include livestock animals, companion animals, and laboratory animals. Non-human subjects also include non-human primates as well as rodents, such as, but not limited to, a rat or a mouse. Non-human subjects also include, without limitation, chickens, horses, cows, pigs, goats, dogs, cats, guinea pigs, hamsters, mink, and rabbits.

As used herein “in vitro” is in cell culture and “in vivo” is within the body of a subject. As used herein, “isolated” refers to material that has been either removed from its natural environment (e.g., the natural environment if it is naturally occurring), produced using recombinant techniques, or chemically or enzymatically synthesized, and thus is altered “by the hand of man” from its natural state.

The above description of the present invention is not intended to describe each disclosed embodiment or every implementation of the present invention. The description exemplifies illustrative embodiments. In several places throughout the application, guidance is provided through lists of examples, which examples can be used in various combinations. In each instance, the recited list serves only as a representative group and should not be interpreted as an exclusive list. For any method disclosed herein that includes discrete steps, the steps may be conducted in any feasible order. And, as appropriate, any combination of two or more steps may be conducted simultaneously.

Unless otherwise indicated, all numbers expressing quantities of components, molecular weights, and so forth used in the specification and claims are to be understood as being modified in all instances by the term “about.” Accordingly, unless otherwise indicated to the contrary, the numerical parameters set forth in the specification and claims are approximations that may vary depending upon the desired properties sought to be obtained by the present invention. At the very least, and not as an attempt to limit the doctrine of equivalents to the scope of the claims, each numerical parameter should at least be construed in light of the number of reported significant digits and by applying ordinary rounding techniques. Notwithstanding that the numerical ranges and parameters setting forth the broad scope of the invention are approximations, the numerical values set forth in the specific examples are reported as precisely as possible. All numerical values, however, inherently contain a range necessarily resulting from the standard deviation found in their respective testing measurements. Also herein, the recitations of numerical ranges by endpoints include all numbers subsumed within that range (e.g., 1 to 5 includes 1, 1.5, 2, 2.75, 3, 3.80, 4, 5, etc.).

Where a range of values is provided, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges may independently be included in the smaller ranges, and are also encompassed within the invention, subject to any specifically excluded limit in the stated range. Where the stated range includes one or both of the limits, ranges excluding either or both of those included limits are also included in the invention.

All headings are for the convenience of the reader and should not be used to limit the meaning of the text that follows the heading, unless so specified.

The term “and/or” means one or all of the listed elements or a combination of any two or more of the listed elements. Unless otherwise specified, “a,” “an,” “the,” and “at least one” are used interchangeably and mean one or more than one.

The terms “comprises” and variations thereof do not have a limiting meaning where these terms appear in the description and claims.

The words “preferred” and “preferably” refer to embodiments of the invention that may afford certain benefits, under certain circumstances. However, other embodiments may also be preferred, under the same or other circumstances. Furthermore, the recitation of one or more preferred embodiments does not imply that other embodiments are not useful, and is not intended to exclude other embodiments from the scope of the invention.

Embodiments of the present disclosure are further described by the following examples. It should be recognized that variations based on the inventive features are within the skill of the ordinary artisan, and that the scope of the disclosure herein should not be limited by the examples. It is to be understood that the particular examples, materials, amounts, and procedures are to be interpreted broadly in accordance with the scope and spirit of the invention as set forth herein. To properly determine the scope of the present disclosure, an interested party should consider the claims herein, and any equivalent thereof.

### EXAMPLES

### Example 1

### Calixarene OTX-008 Targets Human Galectin-1 As an Allosteric Inhibitor of Carbohydrate Binding

With (¹⁵)N-(¹)H HSQC NMR spectroscopy, this example demonstrates that galectin-1 is a molecular target of OTX-008. OTX-008 targets galectin-1 at a site away from this lectin's carbohydrate binding site and thereby attenuates lactose binding to the lectin. This example also demonstrates, using flow cytometry and agglutination assays, that OTX-008 attenuates binding of galectin-1 to cell surface glycans, and that it inhibits cell proliferation in a dose-dependent manner that is correlated with the cellular expression of the lectin. This example shows that OTCX-008 targets galectin-1 as an allosteric inhibitor of glycan/carbohydrate binding.

### Materials and Methods

Chemicals. OTX-008 (also referred to herein as calix[4]arene 00018, 0118, and TX008) was manufactured as previously described (WO 2006/042104; Chen et al., 2006, J Med Chem; 49(26):7754-65; and Dings et al., 2006, J Natl Cancer Inst; 98(13):932-936). Purity of OTX-008 is >95% as determined by chromatography and high accuracy mass spectrometry.

Galectin-1 preparation. Uniformly ¹⁵N-labeled human galectin-1 (gal-1) was expressed in BL21 (DE3) competent cells (Novagen), grown in minimal media, purified over a B lactose affinity column, and further fractionated on a gel filtration column, as described previously (Nesmelova et al., 2008, Biomol NMR Assignments; 2(2):203-205). Typically, 44 milligrams of purified protein were obtained from 1 liter of cell culture. The purity of the final sample was quantified by using the Biorad protein assay and was checked for purity by using SDS PAGE. Functional activity of the purified protein was assessed by a T-cell death assay (Nesmelova et al., 2008, Biomol NAR Assignments; 2(2):203-205).

FITC-Galectin-1 preparation. Gal-1 was conjugated with fluorescein isothiocyanate (FITC) using a FITC:protein molar ratio of 10:1. Gal-1 (2 mg/ml) was dissolved in 500 μM of 20 mM potassium phosphate buffer, pH 7.4, followed by addition of approximately 50 μL FITC (10 mg/ml) that had been dissolved in 0.1 mM sodium bicarbonate. The resulting lower pH (approximately pH 8) ensured selective labeling at the N-terminal amine group of the protein (Hungerford et al., 2007, Photochem Photobiol Sci; 6(2):152-158; and Hermanson, 2008, Bioconjugate Techniques, 2^{nd} ed.; Academic Press: Burlington, Mass. Part 1, 1202 pp). This solution was then mixed thoroughly and incubated at room temperature (22° C.) for 18 hours in the dark. During the course of the reaction, the mixture was gently vortexed 3 or 4 times. The resulting FITC-labeled protein was separated from unbound dye by filtration using an Amicon Ultra cellulose filter (Millipore, 10 kDa cut-off). MALDI-TOF MS demonstrated the addition of the 389 Da FITC label to galectin-1, and suggested >90% labeling efficiency.

Heteronuclear NMR Spectroscopy. Uniformly ¹⁵N-labeled recombinant gal-1 was dissolved at a concentration of 1 mg/ml in 20 mM potassium phosphate buffer at pH 7.0, 50 μM EDTA, made up using a 95% H₂O/5% D₂O mixture. ¹H-¹⁵N HSQC NMR experiments were used to investigate binding of OTX-008 to ¹⁵N-labeled gal-1. ¹H and ¹⁵N resonance assignments for gal-1 were previously reported (Nesmelova et al., 2008, Biomol NMR Assignments; 2(2):203-205; and Nesmelova et al., “Understanding galectin-structure-function relationships to design effective antagonists.” In Galectins; Klyosov A. A., Witczak, Z. J.; Platt D., Eds.; John Wiley & Sons: Hoboken, N.J., 2008; pp 33-69).

NMR experiments were carried out at 30° C. on a Varian Unity Inova 600-MHz spectrometer equipped with an H/C/N triple-resonance probe and x/y/z triple-axis pulse field gradient unit. A gradient sensitivity-enhanced version of two-dimensional ¹H-¹⁵N HSQC was applied with 256 (t1)×2048 (t2) complex data points in nitrogen and proton dimensions, respectively (Nesmelova et al., 2008, Biomol NMR Assignments; 2(2):203-205). Raw data were converted and processed by using NMRPipe (Delaglio et al., 1995, J Biomol NMR; 6(3):277-293) and were analyzed by using NMRview (Johnson, 2004, Methods Mol Biol; 278:313-352).

Flow Cytometry. Male and female gal-1 null mice (Jackson Laboratory) were provided water and standard chow ad libitum and were maintained on a 12-hour light/dark cycle prior to experiments that were approved by the University of Minnesota Research Animal Resources Ethical Committee. For FACS experiments, spleens from these mice (6-10 weeks old) were harvested and non-enzymatically disrupted by shear force to yield single-cell suspensions (Dings et al., 2011, Clin Cancer Res; 17(10):3134-3145). Cell suspensions were prepared in Hanks's balanced solution. Red blood cells were lysed in ACK (Lonza Walkersville) for 5 minutes on ice and suspensions were filtered through nylon mesh. Spleen cells were then washed and incubated with monoclonal antibodies as indicated for 40 minutes on ice. After an additional washing step, various concentrations of gal-1 (with or without OTX-008) were added and incubated for 30 minutes on ice. Prior to FACS analysis, cell suspensions were washed once more and analyzed by multi-parameter flow cytometry on a LSR II flow cytometer (BD Biosciences) using Flowjo software (Tree Star, Inc.) (Dings et al., 2011, Clin Cancer Res; 17(10):3134-3145).

Agglutination Assay. Jurkat E6.1 cells (2×10⁶/200 μL RPMI 1640 medium) agglutination was induced by 1 μM gal-1 and were incubated on plastic round bottom chamber slides (Nunc, Naperville, Ill., USA) in medium alone or with 20 μM OTX-008 at 22° C. Subsequently the cells were analyzed for agglutination with an Axiovert 10 inverted phase microscope equipped with a digital camera system (Zeiss, Germany), and digitally analyzed and differentially quantified by morphometric analysis, as described earlier (Dings et al., 2008, Cancer Lett; 265(2):270-280; and Dings et al., 2010, Bioconjugate Chem; 21(1):20-27).

Cell lines. HEP2, SQ20B, HT29, DU145, and SKHEP1 cells were obtained from the ATCC (Rockville, Md., USA). COL0205-S cells were obtained from the National Cancer Institute collection. COL0205-R cells were developed from COL0205 cells with resistance ingenol-3-angelate protein kinase C modulator and displayed cross resistance to the protein kinase Cβ inhibitor enzastaurin (LY317615-HCl, Eli Lilly) (Serova et al., 2010, Mol Caner Ther; 9(5):1308-1317; and Ghoul et al., 2009, Cancer Res; 69(10):4260-4269). Cells were grown as monolayers in RPMI medium supplemented with 10% fetal calf serum (Invitrogen, Cergy-Pontoise, France), 2 mM glutamine, 100 emits/mL penicillin and 100 μg/mL streptomycin at 37° C. in a humidified 5% CO₂ atmosphere, and regularly checked for the absence of Mycoplasma.

Cell viability assay. Cell viability (i.e. cell proliferation and/or cytotoxicity) was determined using the MTT assay (3-[4,5-dimethylthiazol-2-yl]-2,5-diphenyltetrazolium bromide; Sigma, Saint-Quentin Fallavier, France). The conversion of yellow water-soluble tetrazolium MTT into purple insoluble formazan is catalyzed by mitochondrial dehydrogenases and used to estimate the number of viable cells. In brief, cells were seeded in 96-well tissue culture plates at a density of 2×10³ cells/well. After 72 h drug exposure and a 48 h wash-out period, cells were incubated with 0.4 mg/mL MTT for 4 h at 37° C. After incubation, the supernatant was discarded, insoluble formazan precipitates were dissolved in 0.1 mL of DMSO and the absorbance was measured at 560 nm by use of a microplate reader (Thermo, France). Wells with untreated cells or with drug-containing medium without cells were used as positive and negative controls respectively. IC₅₀ was determined as half the maximal inhibitory drug concentration for each cell line.

Real-Time reverse transcriptase PCR(RT-PCR). The theoretical and practical aspects of real-time quantitative RT-PCR using the ABI Prism 7900 Sequence Detection System (Perkin-Elmer Applied Biosystems, Foster City, Calif., USA) have been described in detail elsewhere (Bieche et al., 2001, Oncogene; 20(56):8109-8115). Results were expressed as n-fold differences in target gene expression relative to the TBP gene (an endogenous RNA control) and relative to a calibrator (1× sample), consisting of the cell line sample from the tested series with the smallest amount of target gene mRNA. Experiments were performed in duplicate.

Western blot analysis. Cells were lysed in buffer containing 50 mM HEPES (pH 7.6), 150 mM NaCl, 1% Triton X-100, 2 mM sodium vanadate, 100 mM NaF, and 0.4 mg/mL phenylmethylsulfonyl fluoride. Equal amounts of protein (30 μg/lane) were subjected to SDS-PAGE and transferred to nitrocellulose membranes. Membranes were blocked with 5% milk in 0.05% Tween 20/phosphate-buffered saline (PBS) and incubated with the primary antibody overnight. Membranes were then washed and incubated with the secondary antibody conjugated to horseradish peroxidase. Bands were visualized using the enhanced chemiluminescence Western blotting detection system. Densitometric analysis was performed under conditions that yielded a linear response. The polyclonal anti-galectin-1 antibodies were purchased from Abcam, Paris, France and generated as described (Rubenstein et al., 2004, Cancer Cell; 5(3):241-251; and Juszczynski et al., 2007, Proc Natl Acad Sci USA; 104(32):13134-13139). All antibodies were used at a 1:1000 dilution.

### Results

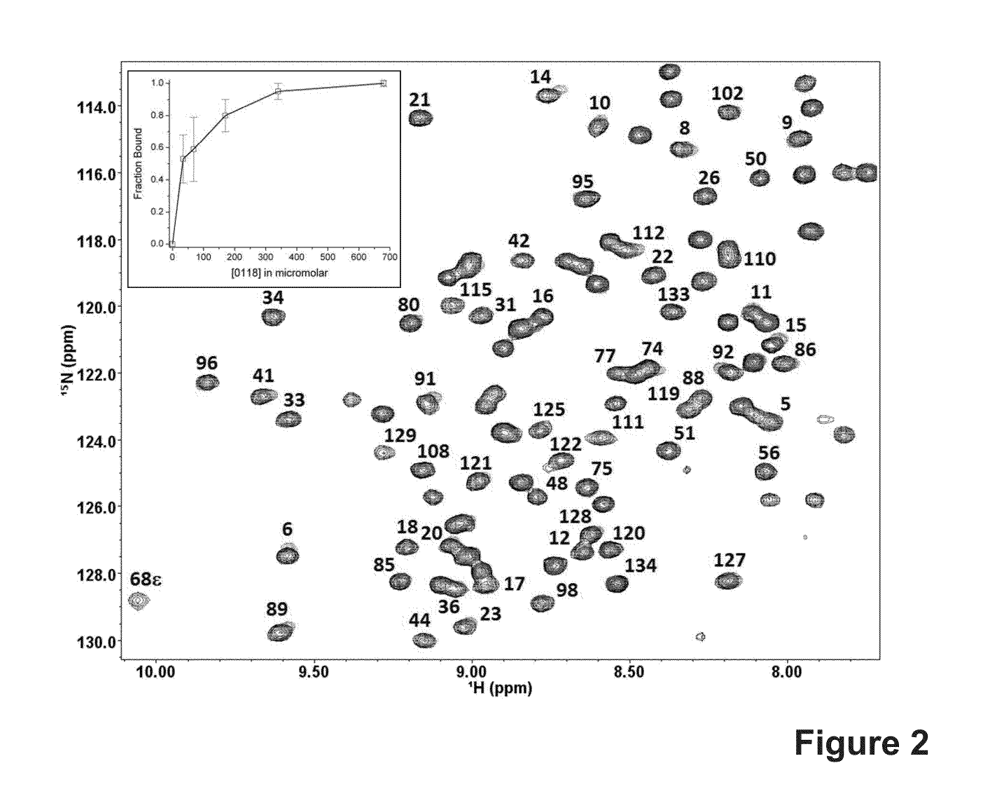

FIG. 2 shows an overlay of two ¹⁵N-¹H HSQC NMR spectra of uniformly ¹⁵N-labeled galectin-1 (¹⁵N-gal-1), one in the absence and one in the presence of 400 μM OTX-008. While some resonances are not perturbed by the presence of OTX-008, others are differentially reduced in intensity (apparent resonance broadening) and chemically shifted. This observation alone indicates that OTX-008 interacts with gal-1. Moreover, these NMR spectral effects demonstrate that the binding exchange between OTX-008 and gal-1 occurs on the intermediate chemical shift time scale.

Although an accurate equilibrium ligand dissociation constant, K_{d} cannot always be determined from data such as these, a plot of OTX-008-induced intensity changes averaged over all resonances vs. the concentration of OTX-008 does yield a saturable binding curve (inset of FIG. 2). The inset illustrates the plotted the concentration of unbound or free OTX-008 ligand by subtracting the estimated fraction bound from the total concentration of OTX-008 added to the solution, e.g. the total concentration of OTX-008 at 50% was 60 μM, and since the total gal-1 monomer concentration was 60 μM, 30 μM would be bound. In this instance, there would be also 30 μM OTX-008 free in solution as plotted. This mid-point value of 30 μM, therefore, is a reasonable estimate of the K_{d} value, and is consistent with the observation that OTX-008 binding to gal-1 occurs in the intermediate exchange regime on the NMR chemical shift time scale, where the equilibrium dissociation constant, K_{d}, for ligand binding should fall in the range of ˜5 μM to ˜100 μM. Ligand binding with a K_{d} value greater than ˜100 μM would be observed as fast exchange on the chemical shift time, whereas a K_{d} value less than ˜1 μM would be observed as slow exchange on the chemical shift time scale. Note that these ranges are only approximate.

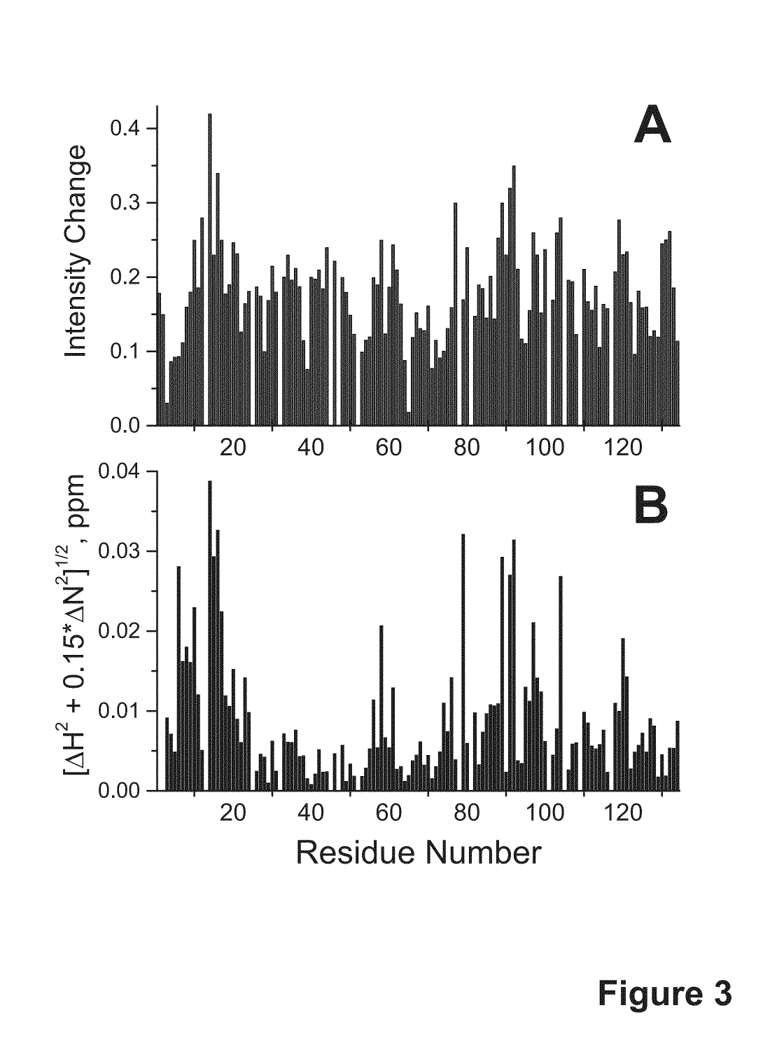

Because the OTX-008-gal-1 binding event falls in the intermediate exchange regime, FIG. 3A shows a resonance broadening map (Miller et al., 2009, Glycobiology; 19(9):1034-1945) in which differential broadening is shown relative to resonance intensities for the gal-1 free state. Fractional changes are calculated by subtracting from one the intensity of a given HSQC cross-peak divided by that in pure gal-1 at the same protein concentration. A value of 1 indicates that a resonance is no longer apparent, and a value of zero indicates no change in resonance intensity. Although the degree of resonance broadening depends on several factors, including the strength of ligand binding and chemical shift differences between unbound and bound states, a resonance broadening map developed at the initial stages of the ligand titration provides relatively good insight into where on the protein the ligand interacts. In this regard, the interpretation of an HSQC broadening map is similar to that of the well-known HSQC chemical shift map (Rajagopal et al., 1997, Protein Sci; 6(12):2624-2627), i.e. those resonances that are broadened the most are associated with that site(s) of interaction on gal-1. However, since resonances in the intermediate exchange regime are chemically shifted as they are broadened (some more than others) and this is correlated with the chemical shift difference between bound and unbound states, FIG. 3B shows a HSQC chemical shift map, also taken at the same initial point in the titration before resonances are too highly broadened and disappear from the spectrum.

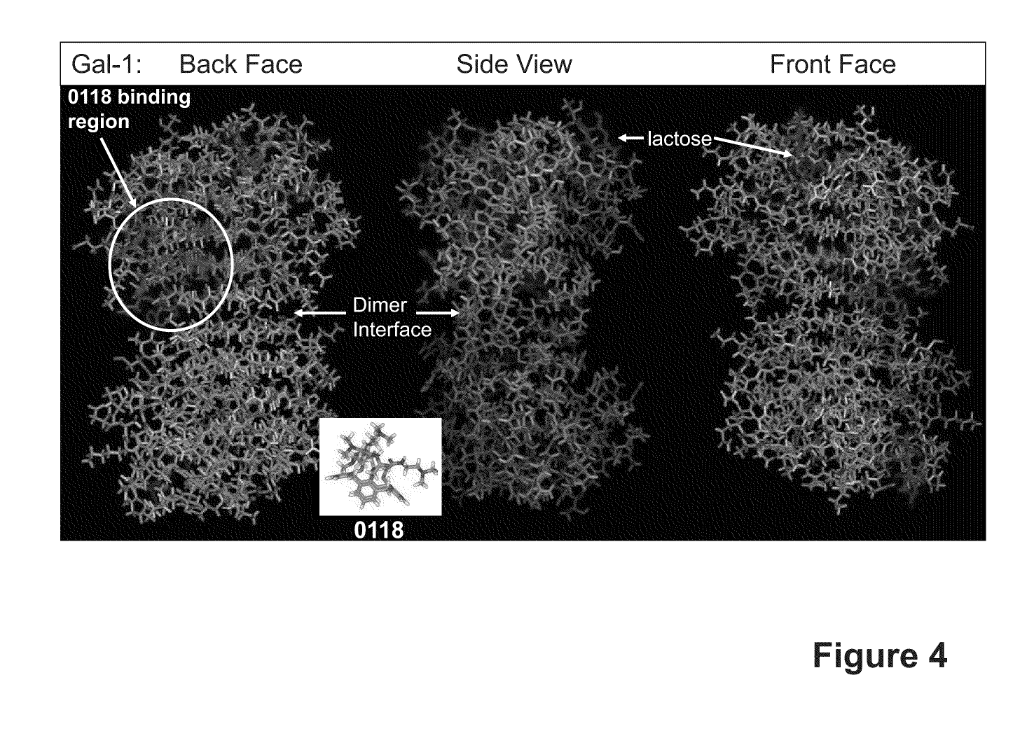

Both broadening and chemical shift maps show similar trends and essentially indicate which gal-1 residues are primarily affected by OTX-008 binding, and therefore, they indicate the region on gal-1 where OTX-008 is most likely to interact (FIG. 4). Gal-1 has a β-sandwich structure that is comprised of 11β-strands, with the front face β-sheet (anti-parallel running β-strands 1, 3, 10, 4, 5, and 6) containing the lactose binding site, and the back face β-sheet comprising β-strands 7, 8, 9, 2, and 11. β-strands 1 and 11 interact to form the gal-1 dimer interface. The HSQC resonance broadening and chemical shift maps (FIG. 3) suggest that the primary site for OTX-008 interaction is at the back face of gal-1 in the region formed primarily by residues 6-10, 14-17, and 89-92. Other residues also perturbed by OTX-008 binding are mostly proximal to these residues. For example, L17 is proximal to M120, and F91 is proximal to T97 which is proximal to Y104.

This region is highlighted (circle in left panel of FIG. 4), along with some other proximal residues, in one of the monomer subunits of the gal-1 dimer (PDB access code 1gzw). The chemical structure of OTX-008 is shown to scale in the insert at the bottom of the figure. The panel at the right in FIG. 4 shows the front face of gal-1 with bound lactose in blue, and the middle panel shows a side view where it is apparent that most perturbations are observed at residues on the back side of gal-1. Nevertheless, by perturbing this network of residues at the back face of gal-1, OTX-008 binding consequently perturbs residues at the front face of gal-1 where carbohydrate ligands bind. In this regard, residues N56, I58, and N61, along with E74 and V76, are indirectly perturbed by OTX-008 binding. This may be explained by considering that Y104 is proximal to E74 and V76 which lie above the carbohydrate ligand, while I89 is proximal to N61 through the β-sandwich. Moreover, M120 is proximal to F79 which interacts with I58, also within the hydrophobic interior of the β-sandwich. In other words, it appears that when OTX-008 binds at the back face of the β-sandwich, effects are seen at the front face carbohydrate binding site.

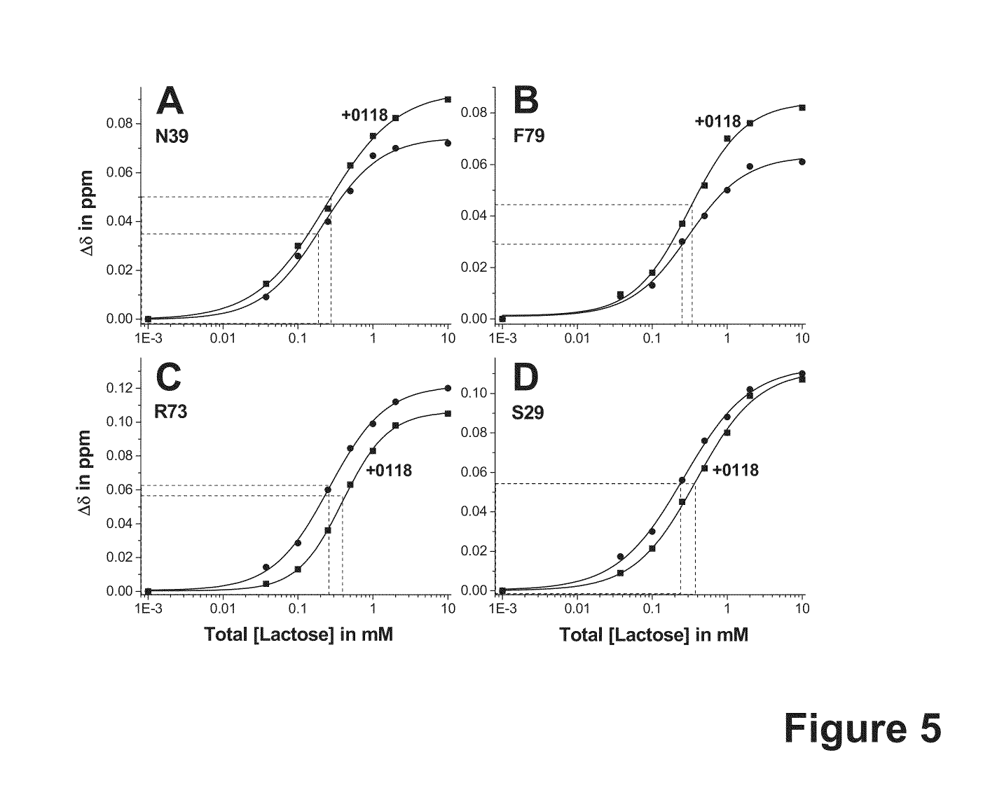

It should be noted that the side chains of N56 and N61 form hydrogen bonds with OH groups from the carbohydrate ligand (Lopez-Lucendo et al., 2004, J Mol Biol; 343(4):957-970), and OTX-008 binding-induced perturbations to these (and other) residues likely would have influence on carbohydrate ligand binding. In fact, lactose binding to gal-1 is attenuated in the presence of OTX-008. FIG. 5 shows ¹⁵N-gal-1 (100 μM) HSQC-derived lactose binding curves acquired in the absence and presence of OTX-008 (gal-1:OTX-008 molar ratio of approximately 1:6). For each of the four residues shown (S29, N39, R73, and F79), curves acquired with gal-1 in the presence of OTX-008 (compared to those in the absence of OTX-008) are shifted to higher concentrations of lactose, indicating weaker ligand binding to the lectin. While the average K_{d} value for binding of lactose to Gal-1 is 100+40 μM in the absence of OTX-008, it is 290+55 μM in the presence of OTX-008. These K_{d} values are the result of averaging of individual K_{d} values determined for the 20 top lactose-induced gal-1 shifted resonances.

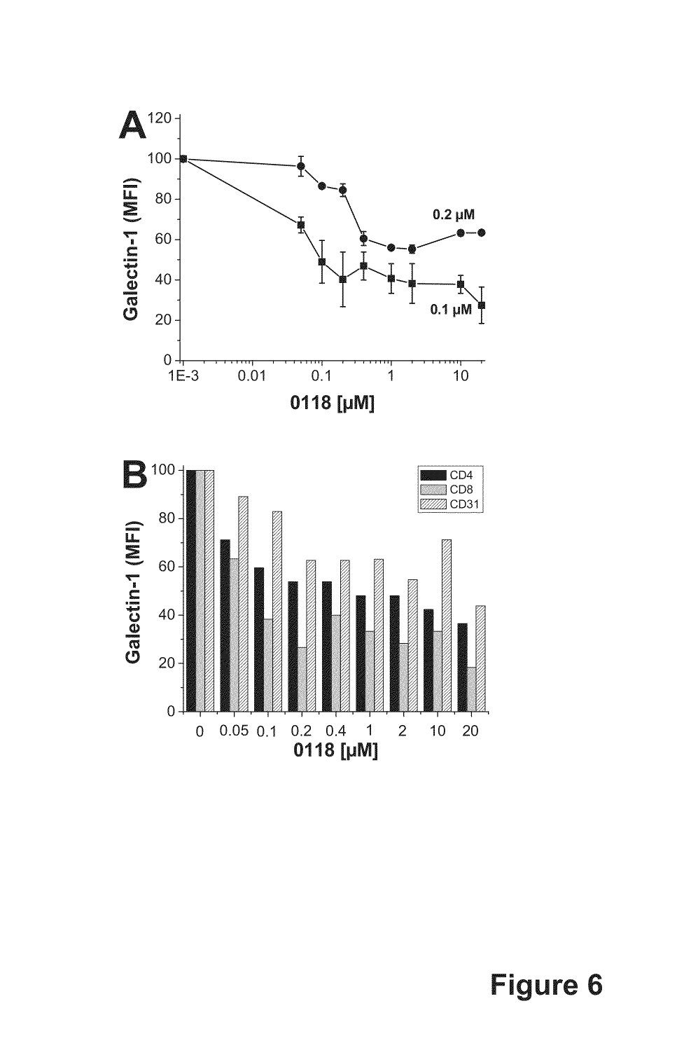

OTX-008-Induced attenuation of carbohydrate (lactose) binding to gal-1 is supported on the cellular level by three lines of evidence. First, results from flow cytometry indicate that FITC-labeled gal-1 (0.1 μM or 0.2 μM) binding to natural glycans on splenocytes is significantly attenuated as the concentration of OTX-008 is increased (FIG. 6A). The effect is de-convoluted with respect to leukocytes (CD4+ and CD8+ cells) and endothelial cells (CD31+) in FIG. 6B. In these cell types, OTX-008 effectively attenuates binding of FITC-gal-1 to cell surface glycans in a dose dependent manner. At 20 μM OTX-008, binding of 0.1 μM gal-1 to CD4+ (or CD8+) and CD31+ cells is ˜80% and ˜50% inhibited, respectively. At 100 μM OTX-008, gal-1 binding to CD4+ (or CD8+) and CD31+ cells is 100% and ˜70% inhibited, respectively. In this regard, gal-1 binding is inhibited in a dose dependent manner, i.e. a higher concentration of OTX-008 is required to inhibit the binding of gal-1 to cell surface glycans when the lectin concentration is 0.2 μM vis-à-vis 0.1 μM.

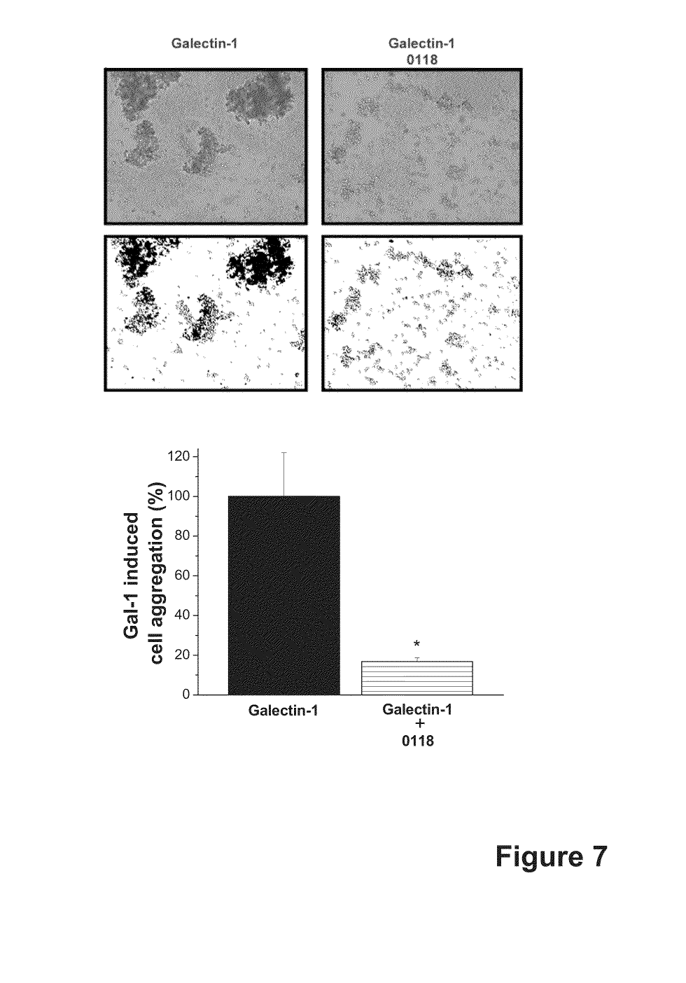

Additional evidence that carbohydrate binding to gal-1 is attenuated by OTX-008 binding comes from the agglutination assay (FIG. 7). Since agglutination occurs by galectin-1-mediated cross-linking of cell surface glycans on cells (Levroney et al., 2005, J Immunol; 175(1):413-420), attenuated agglutination in the presence of OTX-008 can be explained by reduced glycan binding by OTX-008-bound gal-1. At a gal-1 concentration that induces ˜50% cell agglutination (1 μM), FIG. 7A (panels at the left) shows formation of large aggregates as expected in the absence of OTX-008. Upon addition of OTX-008 (20 μM) (panels at the right in FIG. 7A), it is evident that agglutination is highly attenuated. These data were digitally quantified by pixilation, as illustrated in the lower panels to FIG. 7A. These results are presented in bar graph format in FIG. 7B, which indicates that the presence of OTX-008 at 20 μM inhibits cell agglutination by about 80%.

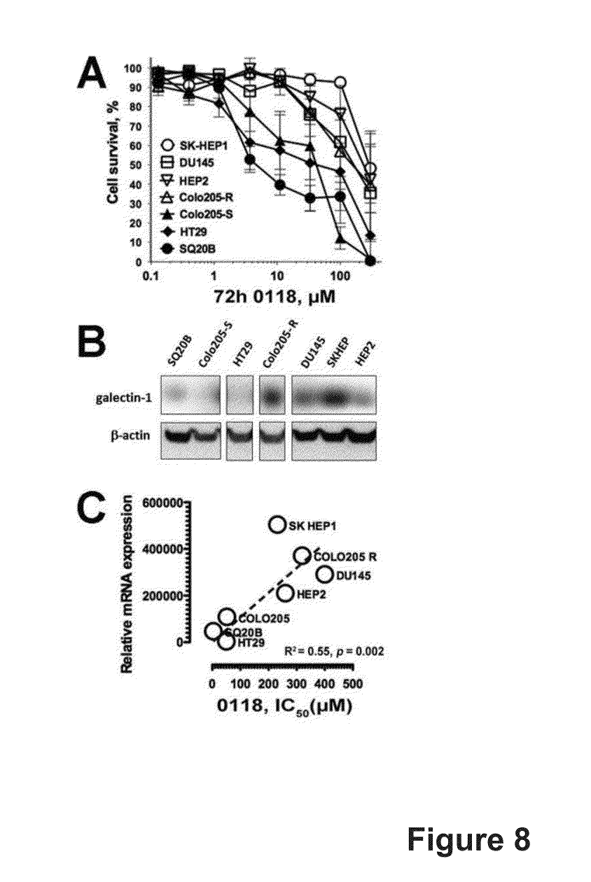

The concentration responsiveness from OTX-008 in these flow cytometry and agglutination experiments with gal-1 parallels that observed in a cell viability assay using a series of human cell lines that express varying amounts of gal-1. FIG. 8A shows the dose-dependent OTX-008 inhibition on several cell types in culture, and FIG. 8B quantifies these results for gal-1 protein production relative to that of β-actin as a control. FIG. 8C shows a scatter plot correlating the IC50 values for OTX-008 with the amount of mRNA gal-1 (LGALS1) expressed in each of those cancer cells. The correlation coefficient (R=0.55, p-value=0.002) is reasonably good, with an inverse correlation indicating that concentrations of OTX-008 required to inhibit 50% of cancer cell growth is proportional to LGALS1 expression.

The disclosure set forth herein demonstrates that one substantial mechanism of action of OTX-008 is via inhibition of gal-1 function. This is, in part, because a correlation between the level of gal-1 expression and the effectiveness of OTX-008 is observed, i.e. greater gal-1 expression requires more OTX-008 to achieve the same biological response. Nevertheless, the disclosure herein also supports the possibility that OTX-008 could also be functioning via an additional mechanism of action. In an embodiment, for example, it is possible that OTX-008 also exerts its effect on cell viability distinct from its effect on gal-1, as set forth elsewhere herein in greater detail.

### Discussion

As described herein, OTX-008 targets gal-1. OTX-008 was designed as a topomimetic of the amphipathic, angiogstatic peptide Anginex (Dings et al., 2007, Acc Chem Res; 40(10):1057-1065). Recent work has reduced the size and peptidic character of the parent peptide 33mer of Anginex to a dibenzofuran-based partial peptide mimetic, 6DBF7, (Mayo et al., 2003, J Biol Chem; 278(46):45746-45752) and then to calixarene compound OTX-008 (Dings et al., 2006, J Natl Cancer Inst 98(13):932-936). However, although the structures of all three compounds are similar in terms of, e.g., being amphipathic and cationic, the actual structural presentation of chemical groups in OTX-008 is quite different from that in Anginex or 6DBF7. The hydrophobic surface of β-sheet-folded Anginex (and 6DBF7) is composed of aromatic (Phe and Trp) and alkyl (Leu, Val, Ile) groups, whereas its hydrophilic, cationic surface is primarily composed of primary amines (Lys). Chemical substituents on the hydrophobic and hydrophilic surfaces of OTX-008 are composed solely of aryl groups and tertiary amines, respectively. Nevertheless, on the molecular level there must be sufficient chemical similarity between Anginex (and 6DBF7) and OTX-008 in order to promote OTX-008 targeting to gal-1 as with Anginex (Thijssen et al., 2006, Proc Natl Acad Sci USA; 103(43):15975-15980).

The greater difference between the chemical surfaces of Anginex and OTX-008 rests in the character of their cationic faces (primary vs. tertiary amines, respectively). Structure-activity relationship information on Anginex indicates that it is the hydrophobic face of the amphipathic peptide that interacts with the surface of gal-1, while the cationic face of Anginex primarily promotes longer range electrostatic effects. In this regard, it is possible that the hydrophobic surface from the calixarene crown of OTX-008 is also more crucial to interaction with gal-1. This possibility is consistent with HSQC mapping data that suggest that the most likely OTX-008 binding site has considerable hydrophobic character defined by e.g. P13, G14, A94, F91, L112, L114, and A116, as well as anionic character with e.g. E15, D92, and E115.

As an antagonist of gal-1, calixarene OTX-008 is unique to the field, primarily because it binds at a site on gal-1 which is located on the back face of the lectin away from its β-galactoside-binding site. Moreover, HSQC-based lactose titrations, as well as flow cytometry and agglutination data, indicate that OTX-008 attenuates binding of gal-1 to lactose and to cell surface glycans. Because of this and the observation that OTX-008 binds gal-1 at a site different from its carbohydrate binding site, in an embodiment, OTX-008 functions as a non-competitive, allosteric inhibitor of gal-1 function, unlike any other known galectin antagonist.

Galectin antagonists reported to date are mostly β-galactoside-analogs and glycomimetics that target the obvious, canonical carbohydrate binding site. Most of these compounds were designed to antagonize galectins 1, 3, 7, 8, and 9 (primarily gal-3) and include aryl O- and S-galactosides and lactosides, carbohydrate-based triazoles and isoxazoles, 3-(1,2,3-triazol-1-yl)-1-thio-galactosides, anomeric oxime ether derivatives of β-galactose (O-galactosyl aldoximes), phenyl thio-β-D-galactopyranoside analogs, thioureido N-acetyllactosamine derivatives, and multi-valent arene thiodigalactoside bis-benzamido analogs with groups on each end of the carbohydrate moiety to interact with arginine residues within the carbohydrate binding domain. Other multivalent inhibitors include functionalized unnatural amino acids (phenyl-bis-alanine and phenyl-tris-alanine) with 2-azidoethyl β-D-galactopyranosyl-(1-4)-β-D-glucopyranoside,10 a bi-lactosylated steroid-based compound, and polymethylene-spaced di-lactoseamine derivatives.

Although some of these carbohydrate-based compounds bind various galectins in vitro with single digit μM Kd values, most bind galectins rather weakly with Kd's>100 μM. Moreover, galectin specificity of any of them remains a significant issue, due primarily to conserved structural homology of β-galactoside binding sites among all galectins. In addition, few of these compounds has been tested in vivo, where issues related to bioavailability are likely to show that these saccharide-based compounds would make for relatively poor therapeutic agents, primarily because simple, low molecular weight carbohydrates are readily subject to clearance and hydrolysis.

In an embodiment, OTX-008 is a therapeutic agent for use against cancer. As a therapeutic agent, calixarene OTX-008 is a significant improvement over both Anginex and 6DBF7, as well as over any carbohydrate-based compound, for multiple reasons. OTX-008 (937 Da) approaches the generally accepted definition of a “small molecule” (i.e. <500 Da). OTX-008 should have better in vivo exposure, because it is non-peptidic and non-carbohydrate, and therefore is non-hydrolyzable. Moreover, because of its aryl-crown structure, OTX-008 should be chemically stable and less likely to be metabolized in vivo than any peptide or carbohydrate-based compound. Preliminary assessment of OTX-008 in a Phase I clinical trial with terminal cancer patients administered daily (initial and current cohort at 1 mg/Kg) via subcutaneous injection indicates the absence of toxicity, relatively long half-life (˜48 hrs), and an apparent stabilization of disease state. In an embodiment, OTX-008 is a promising anti-tumor agent that targets galectin-1 as a first-in-class therapeutic agent.

The binding of OTX-008 to galectin-1 and the effects of this binding will be further characterized. Such studies may for example, any of the assays, cell lines, constructs, and/or galectin-1-associated pathways discussed in PCT/EP2012/055902, which is herein incorporated by reference in its entirety. Such studies may include, for example, the effect of the administration of OTX-008 on tumor survival, growth, and/or metastasis of cancer cell lines including, but not limited to, including, but not limited to HT29, HCT116, COLO205, or HCC2998 (human colon cancer cell lines), MCF7, MDA-MB-435, SKBR3, or ZR-75-1 (human breast cancer cell lines), A2780-1A9, OVCAR3, IGROV1, or SKOV3 (human ovarian cancer cell lines), HOP62 or HOP92 (human lung cancer cell lines), PC3 or DU145 (human prostate cancer cell lines), SCC61, SQ20B, or HEP2 (human squamous cell head and neck cancer cell lines), SK-HEP 1 (a human hepatocellularcarcinoma cancer cell line), or CAKI1 (a human renal cancer cell line). See, for example, Astorgues-Xerri et al., 2009, Molecular Cancer Therapies; Volume 8, Issue 12, Supplement 1, Abstract B166. Such determinations may be include in vitro and/or in vivo assays, including, but not limited to, any of those described herein

Example 1 has also published as Dings et al., “Antitumor agent calixarene 0118 targets human galectin-1 as an allosteric inhibitor of carbohydrate binding,” J Med. Chem. 2012 Jun. 14; 55(11):5121-9; Epub 2012 May 30, which is incorporated by reference herein in its entirety.

The complete disclosure of all patents, patent applications, and publications, and electronically available material (including, for instance, nucleotide sequence submissions in, e.g., GenBank and RefSeq, and amino acid sequence submissions in, e.g., SwissProt, PIR, PRF, PDB, and translations from annotated coding regions in GenBank and RefSeq) cited herein are incorporated by reference. In the event that any inconsistency exists between the disclosure of the present application and the disclosure(s) of any document incorporated herein by reference, the disclosure of the present application shall govern. The foregoing detailed description and examples have been given for clarity of understanding only. No unnecessary limitations are to be understood therefrom. The invention is not limited to the exact details shown and described, for variations obvious to one skilled in the art will be included within the invention defined by the claims.

Sequence Listing Free Text

| | |
|---|---|
| SEQ ID NO: 1 | Amino acid sequence of anginex |
| SEQ ID NO: 2-3 | Amino acid sequences of the (DBF-based mimetic) |
| | 6DBF7 |
| SEQ ID NO: 4 | Amino acid sequence of human Galectin-1 |

What is claimed is:

1. A method of inhibiting a pathway associated with galectin-1 in a mammalian cell, wherein galectin-1 has a front face β-sheet and a back face β-sheet, the method comprising contacting a mammalian cell with an effective amount of a galectin-1-targeting compound, wherein the galectin-1-targeting compound has an affinity for the back face β-sheet of galectin-1.

2. A method of treating cancer in a subject in need thereof, the method comprising administering a therapeutically effective amount of a compound having an affinity for a back face β-sheet of galectin-1 to a subject in need thereof.

3. The method of claim 1, wherein the compound additionally binds to the front face β-sheet.

4. The method of claim 1, wherein the compound does not bind to the front face β-sheet.

5. The method of claim 1, wherein the compound binds to galectin-1 on at least one site other than the carbohydrate binding site.

6. The method of claim 1, wherein the compound attenuates the binding of lactose to the front face β-sheet.

7. The method of claim 1, wherein the compound attenuates the binding of the galectin-1 to one or more cell surface glycans.

8. The method of claim 1, wherein the galectin-1-targeting compound binds to galectin-1 with a K_{d} in the range of about 1 μM to about 100 μM.

9. The method of claim 1, wherein the method is a method of inhibiting galectin-1 activity comprising contacting a cell with a therapeutically effective amount of a galectin-1 targeting compound.

10. The method of claim 1, wherein the compound contacts one or more residues of a galectin-1 selected from residues corresponding to residues 6, 7, 8, 9, 10, 14, 15, 16, 17, 89, 90, 91, and/or 92 of the human galectin-1 sequence set forth in SEQ ID NO:4.

11. The method of claim 1, wherein the compound contacts one or more residues of human galectin-1 selected from residues 6, 7, 8, 9, 10, 14, 15, 16, 17, 89, 90, 91, and/or 92 of the human galectin-1 sequence set forth in SEQ ID NO:4.

12. The method of claim 1, wherein the galectin-1 targeting compound is OTX-008, OTX-008 having the formula

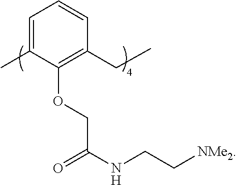

13. The method of claim 1, the method comprising contacting one or more cancerous cells in a subject with cancer with a therapeutically effective amount of the galectin-1 targeting compound.

14. The method of claim 13, wherein the method inhibits growth of the cancer cell, proliferation of the cancer cell, and/or inhibits tumor metastasis.

15. The method of claim 2, wherein the treatment of the cancer includes at least one of slowing the growth of the cancer, inhibiting the spread of a tumor associated with the cancer, inhibiting the spread of one or more metastases associated with the cancer, reducing the size of a tumor associated with the cancer, and/or inhibiting the recurrence of cancer treated previously.

16. The method of claim 2, wherein the compound is administered by at least one route selected from the group consisting of intravenously, subcutaneously, intradermally, parenterally, and intramuscularly.

17. The method of claim 2 wherein the compound is administered in a single dose.

18. The method of claim 2 wherein the compound is administered in two or more doses.

19. The method of claim 2, wherein the compound is administered in combination with at least one other mode of therapy.

20. The method of claim 19, wherein the at least one other mode of therapy compound is selected from the group consisting of radiotherapy, chemotherapy, and surgery and combinations thereof.

21. The method of claim 2, wherein the therapeutically effective amount of therapeutically effective amount of the compound comprises a pharmaceutical composition.

22. The method of claim 2, wherein the compound comprises OTX-008, OTX-008 having the formula

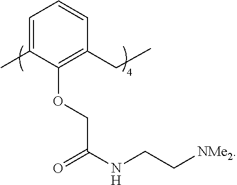

23. The method of claim 21, wherein the pharmaceutical composition is administered by at least one route selected from the group consisting of intravenously, subcutaneously, intradermally, parenterally, and intramuscularly.

24. The method of claim 2, wherein the cancer is selected from the group consisting of ovarian cancer, squamous cell carcinoma, a cancer of the digestive system, stomach cancer, liver cancer, colon cancer, a cancer of the thyroid, a cancer of the endometrium, adenocarcinoma of the endometrium, uterine cancer, uterine adenocarcinoma, a uterine smooth muscle tumor, breast cancer, prostate cancer, bladder cancer, a head cancer, a neck cancer, a glioma, a kidney cancer, pancreatic cancer, pancreatic ductal adenocarcinoma, nonsmall-cell lung cancer, and melanoma.

25. The method of claim 2, wherein the galectin-1-targeting compound is administered in a dose selected from the group consisting of about 1 microgram to about 1,000 micrograms, about 25 micrograms to about 500 micrograms, about 50 to about 250 micrograms, about 2 micrograms to about 100 micrograms, about 5 micrograms to about 75 micrograms, about 10 micrograms to about 50 micrograms, and about 20 micrograms to about 40 micrograms.

26. The method of claim 22, wherein the OTX-008 is administered in a dose selected from the group consisting of about 1 microgram to about 1,000 micrograms, about 25 micrograms to about 500 micrograms, about 50 to about 250 micrograms, about 2 micrograms to about 100 micrograms, about 5 micrograms to about 75 micrograms, about 10 micrograms to about 50 micrograms, and about 20 micrograms to about 40 micrograms.

27. A method of allosterically inhibiting the binding of a glycan carbohydrate to galectin-1 at a site other than the canonical β-galactoside carbohydrate binding site, the method comprising contacting the galectin-1 with OTX-008, OTX-008 having the formula

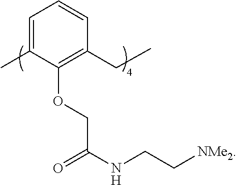

28. A method of inhibiting proliferation in galectin-1 positive cells, the method comprising contacting the galectin-1 positive cells with OTX-008, OTX-008 having the formula

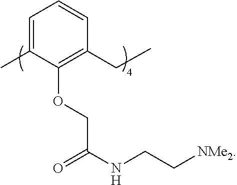

29. A method of inhibiting the binding of galectin-1 to cell surface glycans and/or inhibiting galectin-1 mediated cell agluttination, the method comprising contacting the cells with OTX-008, OTX-008 having the formula

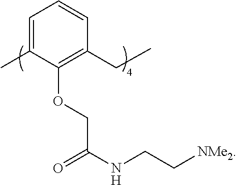

30. A method of inhibiting the galectin-1/semaphorin-3A system in a cancer cell, the method comprising contacting the cell with an effective amount of a galectin-1-targeting compound, wherein the compound binds to galectin-1 on at least one site other than the carbohydrate binding site.
